(19) United States
(12) Patent Application Publication
(10) Pub. No.: US 2024/0203530 A1
(43) Pub. Date: Jun. 20, 2024

(54) MACHINE LEARNING TECHNIQUES TO DETERMINE BASE METHYLATIONS

(71) Applicant: Centre for Novostics, Shatin (HK)

(72) Inventors: Yuk-Ming Dennis Lo, Homantin (CN); Rossa Wai Kwun Chiu, Shatin (CN); Kwan Chee Chan, Jordan (CN); Peiyong Jiang, Pak Shek Kok (CN); Xi Hu, Pak Shek Kok (CN)

(21) Appl. No.: 18/542,251

(22) Filed: Dec. 15, 2023

Related U.S. Application Data
(60) Provisional application No. 63/433,253, filed on Dec. 16, 2022.

(51) Int. Cl. G16B 40/20; G06N 20/00; G16B 20/20

(52) U.S. Cl. CPC G16B 40/20; G06N 20/00; G16B 20/20

(57) ABSTRACT

Systems and methods determine based methylations in analyzing nucleic acid molecules. Embodiments may use kinetic signals produced by a DNA polymerase during single-molecule sequencing. The use of kinetic signals of adaptor sequences may allow for determining the methylation patterns proximal to the ends of a DNA fragment. Deep learning models that can capture local and global signal patterns can be trained to detect the base methylations using these features, with improved performance. Deep learning models may include a convolutional neural network that preferentially captures the features of local signal patterns, with the integration of a transformer model that preferentially captures the features of global signal patterns. The improved performance in the determination of base methylation may lead to more accurate diagnoses of subjects. Accurate measurement of DNA methylation may have several other clinical applications.

### CROSS-REFERENCE TO RELATED APPLICATION

The present application claims the benefit of priority of U.S. Provisional Application No. 63/433,253, filed Dec. 16, 2022, the entire contents of which are incorporated herein by reference for all purposes.

### BACKGROUND

DNA methylation is an epigenetic mechanism by which a methyl group is added to a DNA base. For example, a methyl group may be covalently added onto the 5th position of cytosine to form 5-methylcytosine. Methylation has been found on cytosines, adenines, thymines and guanines, such as 5 mC (5-methylcytosine), 6 mA (N6-methyladenine), 4 mC (N4-methylcytosine), 5 hmC (5-hydroxymethylcytosine), 5 fC (5-formylcytosine), 5 caC (5-carboxylcytosine), 1 mA (N1-methyladenine), 3 mA (N3-methyladenine), 7 mA (N7-methyladenine), 3 mC (N3-methylcytosine), 2 mG (N2-methylguanine), 6 mG (O6-methylguanine), 7 mG (N7-methylguanine), 3 mT (N3-methylthymine), and 4 mT (O4-methylthymine).

DNA methylation plays important biological roles, for example, silencing retroviral elements, regulating tissue-specific gene expression, genomic imprinting, X chromosome inactivation, tumorigenesis, and modulating many other diseases (Moore et al. Neuropsychopharmacology. 2013;38:23-38). DNA methylation occurring in different genomic regions may exert different influences on gene activities based on the underlying genetic sequence. Hence, the accurate measurement of DNA methylation would have numerous clinical implications.

The accurate measurement of methylomic modification on DNA molecules would have numerous clinical implications. One widely used method to measure DNA methylation is through the use of bisulfite sequencing (BS-seq) (Lister et al., 2009; Frommer et al., 1992). In this approach, DNA samples are first treated with bisulfite which converts unmethylated cytosine (i.e. C) to uracil. In contrast, the methylated cytosine remains unchanged. The bisulfite modified DNA is then analyzed by DNA sequencing. In another approach, following bisulfite conversion, the modified DNA is then subjected to polymerase chain reaction (PCR) amplification using primers that can differentiate bisulfite converted DNA of different methylation profiles (Herman et al., 1996). This latter approach is called methylation-specific PCR.

One disadvantage of such bisulfite-based approaches is that the bisulfite conversion step has been reported to significantly degrade the majority of the treated DNA (Grunau, 2001). Another disadvantage is that the bisulfite conversion step would create strong CG biases (Olova et al., 2018), resulting in the reduction of signal-to-noise ratios typically for DNA mixtures with heterogeneous methylation states. Furthermore, bisulfite sequencing would not be an ideal method to sequence long DNA molecules because of the degradation of DNA during bisulfite treatment.

Systems and methods described herein improve accuracy and efficiency of detecting DNA methylation. Systems and methods may avoid chemical conversion steps to detect methylation. Systems and methods may also be less expensive than other systems and methods.

### BRIEF SUMMARY

In this disclosure, enhanced systems and methods determine base methylation in analyzing nucleic acid molecules. Embodiments may use kinetic signals produced by a DNA polymerase during single-molecule sequencing. Methods described herein may include using features derived from kinetic signals of sequencing. These features may include the pulse width of an optical signal or electrical signal from sequencing bases, the interpulse duration of bases, and the identity of the bases, which can be generated from DNA molecules of interest extracted from an organism (e.g., human) and the artificial sequences (adaptors) ligated to DNA molecules of interest. The use of kinetic signals of adaptor sequences may allow for determining the methylation patterns proximal to the ends of a DNA fragment, which may initially not able to be analyzed due to insufficient kinetic signals flanking on the loci close fragment ends.

Machine learning models that can capture local and global signal patterns can be trained to detect the base methylations using these features, with improved performance. Machine learning models may include a convolutional neural network that preferentially captures the features of local signal patterns, with the integration of a transformer model that preferentially captures the features of global signal patterns. The improved performance in the determination of base methylation may lead to more accurate diagnoses of subjects. Accurate measurement of DNA methylation may have several other clinical applications.

These and other embodiments of the disclosure are described in detail below. For example, other embodiments are directed to systems, devices, and computer readable media associated with methods described herein.

A better understanding of the nature and advantages of embodiments of the present invention may be gained with reference to the following detailed description and the accompanying drawings.

### BRIEF DESCRIPTION OF THE DRAWINGS

FIG. 1 shows an example model framework for DNA methylation according to embodiments of the present invention.

FIGS. 2A and 2B show example measurement windows according to embodiments of the present invention.

FIG. 2C is an illustrative graph that demonstrates an example of integrating the results of convolutional layers and positional information of a DNA segment to form an input matrix of downstream layers according to embodiments of the present invention.

FIG. 2D illustrates transformer layers according to embodiments of the present invention.

FIG. 3A shows receiver operating characteristic (ROC) curves comparing models according to embodiments of the present invention.

FIG. 3B shows a table comparing sensitivities at given specificities for different models according to embodiments of the present invention.

FIG. 4A shows receiver operating characteristic (ROC) curves for different models and datasets according to embodiments of the present invention.

FIG. 4B is a graph showing accuracy and subread depth according to embodiments of the present invention.

FIG. 5 is an ROC curve for HK model 2 with using only single strands according to embodiments of the present invention.

FIG. 6 shows the AUC of methylation analysis for CpG sites at positions relative to the nearest end of sequenced fragments for two datasets according to embodiments of the present invention.

FIG. 7 illustrates the protocol for improved performance of the single-stranded model for methylation sites close to the 3′ end according to embodiments of the present invention.

FIG. 8A shows the composition of TET-treated DNA. The amount of 5 hmC, 5 fC, and 5 acC varies with incubation time according to embodiments of the present invention.

FIG. 8B shows the preparation of the 5 hmC detection dataset using ligation according to embodiments of the present invention.

FIG. 8C shows the analytical workflow for 5 mC and 5 hmC detection according to embodiments of the present invention.

FIG. 9A shows an ROC curve of the testing datasets for the 5 xC and 5 hmC detectors according to embodiments of the present invention.

FIG. 9B shows box plots of the modification scores predicted by the 5 hmC detector in the testing dataset according to embodiments of the present invention.

FIG. 10A shows methylation levels measured by different approaches in buffy coat and brain samples across different genomic regions of interest according to embodiments of the present invention.

FIG. 10B shows methylation levels predicted by HK model 2 in human brain samples around transcription start sites (TSS) sites according to embodiments of the present invention.

FIG. 10C shows the correlation of the 5 xC levels in brain samples measured by the HK model 2 and BS-seq according to embodiments of the present invention.

FIG. 10D shows the correlation of the 5 hmC levels (%) in brain samples measured by the HK model 2 and TAB-seq according to embodiments of the present invention.

FIG. 11A is a schematic for preparing the unmethylated and methylated adenine datasets according to embodiments of the present invention.

FIG. 11B shows the IPD distributions in uA and 6 mA datasets according to embodiments of the present invention.

FIG. 11C shows ROC curves of 6 mA detection based on HK model 2 and only the IPD metric according to embodiments of the present invention.

FIG. 11D shows false positive rates of 6 mA detection based on HK model 2 and only the IPD metric according to embodiments of the present invention.

FIG. 11E shows 6 mA methylation levels determined by HK model 2 in non-GATC and GATC contexts in the Dam-treated DNA sample according to embodiments of the present invention.

FIG. 12A shows the IPD distributions in uC and 4 mC datasets according to embodiments of the present invention.

FIG. 12B shows ROC curves of 4 mC detection based on HK model 2 and only the IPD metric according to embodiments of the present invention.

FIG. 13A shows 6 mA methylation levels determined by HK model 2 according to embodiments of the present invention.

FIG. 13B shows de novo motif analysis related to 6 mA modifications according to embodiments of the present invention.

FIG. 14 illustrates sparse and dense signal patterns with a 6 mA modification according to embodiments of the present invention.

FIG. 15A illustrates the distribution of kinetics features in a measurement window between before and after signal normalization in different datasets according to embodiments of the present invention.

FIG. 15B shows the density distributions of kinetic features in different bases of templated DNA on the basis of PacBio Sequel II kit 2.0 according to embodiments of the present invention.

FIG. 16 shows the performance of classifiers of 6 mA using different types of 6 mA signals according to embodiments of the present invention.

FIG. 17 shows the different sensitivities at given specificities for different double-stranded models according to embodiments of the present invention.

FIG. 18 shows different sensitivities at given specificities for different single-stranded models according to embodiments of the present invention.

FIG. 19A shows HCC methylation scores determined by HK model 2 in healthy individuals, HBV carriers, and HCC patients using sequenced DNA molecules with 1 to 6 CpG sites according to embodiments of the present invention.

FIG. 19B shows ROC curves of using HCC methylation score for classifying individuals with and without HCC on the basis of molecules with 1 to 6 CpG sites or at least 7 CpG sites according to embodiments of the present invention.

FIG. 19C shows patterns of 6 mA levels in genomic sites relative to CTCF binding sites

according to embodiments of the present invention.

FIG. 20 is a flowchart of a process for detecting a methylation of a nucleotide in a nucleic acid molecule according to embodiments of the present invention.

FIG. 21 is a flowchart of a process for detecting a methylation of a nucleotide in a nucleic acid molecule according to embodiments of the present invention.

FIG. 22 is a graph of the callable CpG sites versus distance to the nearest end according to embodiments of the present invention.

FIG. 23 shows a workflow for using adaptor sequences to analyze methylation of a site according to embodiments of the present invention.

FIG. 24 is a graph of the performance of the EMA model for determining methylation status of CpG sites within 10 nt of the 5′ end of the DNA fragment according to embodiments of the present invention.

FIG. 25 is a flowchart of a process for detecting a methylation of a nucleotide in a nucleic acid molecule with an adaptor according to embodiments of the present invention.

FIG. 26 is a flowchart of a process for detecting a methylation of a nucleotide in a nucleic acid molecule with an adaptor according to embodiments of the present invention.

FIG. 27 illustrates a measurement system according to embodiments of the present invention.

FIG. 28 is a computer system according to embodiments of the present invention.

### TERMS

A “tissue” corresponds to a group of cells that group together as a functional unit. More than one type of cells can be found in a single tissue. Different types of tissue may consist of different types of cells (e.g., hepatocytes, alveolar cells or blood cells), but also may correspond to tissue from different organisms (mother vs. fetus) or to healthy cells vs. tumor cells. “Reference tissues” can correspond to tissues used to determine tissue-specific methylation levels. Multiple samples of a same tissue type from different individuals may be used to determine a tissue-specific methylation level for that tissue type.

A “biological sample” refers to any sample that is taken from a subject (e.g., a human (or other animal), such as a pregnant woman, a person with cancer or other disorder, or a person suspected of having cancer or other disorder, an organ transplant recipient or a subject suspected of having a disease process involving an organ (e.g., the heart in myocardial infarction, or the brain in stroke, or the hematopoietic system in anemia) and contains one or more nucleic acid molecule(s) of interest. The biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), intraocular fluids (e.g., the aqueous humor), etc. Stool samples can also be used. In various embodiments, the majority of DNA in a biological sample that has been enriched for cell-free DNA (e.g., a plasma sample obtained via a centrifugation protocol) can be cell-free, e.g., greater than 50%, 60%, 70%, 80%, 90%, 95%, or 99% of the DNA can be cell-free. The centrifugation protocol can include, for example, 3,000 g×10 minutes, obtaining the fluid part, and re-centrifuging at for example, 30,000 g for another 10 minutes to remove residual cells. As part of an analysis of a biological sample, a statistically significant number of cell-free DNA molecules can be analyzed (e.g., to provide an accurate measurement) for a biological sample. In some embodiments, at least 1,000 cell-free DNA molecules are analyzed. In other embodiments, at least 10,000 or 50,000 or 100,000 or 500,000 or 1,000,000 or 5,000,000 cell-free DNA molecules, or more, can be analyzed. At least a same number of sequence reads can be analyzed.

“Clinically-relevant DNA” can refer to DNA of a particular tissue source that is to be measured, e.g., to determine a fractional concentration of such DNA or to classify a phenotype of a sample (e.g., plasma). Examples of clinically-relevant DNA are fetal DNA in maternal plasma or tumor DNA in a patient's plasma or other sample with cell-free DNA. Another example includes the measurement of the amount of graft-associated DNA in the plasma, serum, or urine of a transplant patient. A further example includes the measurement of the fractional concentrations of hematopoietic and nonhematopoietic DNA in the plasma of a subject, or fractional concentration of a liver DNA fragments (or other tissue) in a sample or fractional concentration of brain DNA fragments in cerebrospinal fluid.

A “sequence read” refers to a string of nucleotides obtained from any part or all of a nucleic acid molecule. For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)).

Such sequencing can be random sequencing or targeted sequencing (e.g., by using capture probes hybridizing to specific regions or by amplifying certain region, both of which enrich such regions). Example PCR techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000 or 50,000 or 100,000 or 500,000 or 1,000,000 or 5,000,000 sequence reads, or more, can be analyzed.

A “subread” is a sequence generated from all bases in one strand of a circularized DNA template that has been copied in one contiguous strand by a DNA polymerase. For example, a subread can correspond to one strand of circularized template DNA. In such an example, after circularization, one double-stranded DNA molecule would have two subreads: one for each sequencing pass. In some embodiments, the sequence generated may include a subset of all the bases in one strand, e.g., because of the existence of sequencing errors.

An “adaptor” or “adapter” may be an oligonucleotide that is ligated onto an end of a nucleic acid molecule. The nucleic acid molecule may be DNA or RNA. Adaptors may include hairpin adaptors, which are oligonucleotides that ligate to both terminals of one end of a double-stranded DNA molecule. The adaptor may facilitate sequencing techniques, including single molecule real-time sequencing. Some of the nucleotides of the adaptor may be sequenced when sequencing the target nucleic acid molecule.

A “site” corresponds to a single site, which may be a single base position or a group of correlated base positions, e.g., a CpG site. A “locus” may correspond to a region that includes multiple sites. A locus can include just one site, which would make the locus equivalent to a site in that context. Various embodiments can analyze a statistically significant number of loci, e.g., at least 100, 200, 500, 1,000, 5,000, 10,000, 50,000, 100,000, 500,000, 1,000,000, or more loci.

A “methylation status” refers to the state of methylation at a given site. For example, a site may be either methylated, unmethylated, or in some cases, undetermined.

The “methylation index” for each genomic site (e.g., a CpG site) can refer to the proportion of DNA fragments (e.g., as determined from sequence reads or probes) showing methylation at the site over the total number of reads covering that site. A “read” can correspond to information (e.g., methylation status at a site) obtained from a DNA fragment. A read can be obtained using reagents (e.g., primers or probes) that preferentially hybridize to DNA fragments of a particular methylation status at one or more sites. Typically, such reagents are applied after treatment with a process that differentially modifies or differentially recognizes DNA molecules depending on their methylation status, e.g. bisulfite conversion, or methylation-sensitive restriction enzyme, or methylation binding proteins, or anti-methylcytosine antibodies, or single molecule sequencing techniques (e.g. single molecule, real-time sequencing and nanopore sequencing (e.g. from Oxford Nanopore Technologies)) that recognize methylcytosines and hydroxymethylcytosines.

The “methylation density” of a region can refer to the number of reads at sites within the region showing methylation divided by the total number of reads covering the sites in the region. The sites may have specific characteristics, e.g., being CpG sites. Thus, the “CpG methylation density” of a region can refer to the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of cytosines not converted after bisulfite treatment (which corresponds to methylated cytosine) at CpG sites as a proportion of all CpG sites covered by sequence reads mapped to the 100-kb region. This analysis can also be performed for other bin sizes, e.g., 500 bp, 5 kb, 10 kb, 50-kb or 1-Mb, etc. A region could be the entire genome or a chromosome or part of a chromosome (e.g., a chromosomal arm). The methylation index of a CpG site is the same as the methylation density for a region when the region only includes that CpG site. The “proportion of methylated cytosines” can refer the number of cytosine sites, “C's”, that are shown to be methylated (for example unconverted after bisulfite conversion) over the total number of analyzed cytosine residues, i.e. including cytosines outside of the CpG context, in the region. The methylation index, methylation density, count of molecules methylated at one or more sites, and proportion of molecules methylated (e.g., cytosines) at one or more sites are examples of “methylation levels.” Apart from bisulfite conversion, other processes known to those skilled in the art can be used to interrogate the methylation status of DNA molecules, including, but not limited to enzymes sensitive to the methylation status (e.g. methylation-sensitive restriction enzymes), methylation binding proteins, single molecule sequencing using a platform sensitive to the methylation status (e.g. nanopore sequencing (Schreiber et al. Proc Natl Acad Sci 2013; 110: 18910-18915) and by single molecule, real-time sequencing (e.g. that from Pacific Biosciences) (Flusberg et al. Nat Methods 2010; 7: 461-465)).

A “methylome” provides a measure of an amount of DNA methylation at a plurality of sites or loci in a genome. The methylome may correspond to all of the genome, a substantial part of the genome, or relatively small portion(s) of the genome.

A “pregnant plasma methylome” is the methylome determined from the plasma or serum of a pregnant animal (e.g., a human). The pregnant plasma methylome is an example of a cell-free methylome since plasma and serum include cell-free DNA. The pregnant plasma methylome is also an example of a mixed methylome since it is a mixture of DNA from different organs or tissues or cells within a body. In one embodiment, such cells are the hematopoietic cells, including, but not limited to cells of the erythroid (i.e., red cell) lineage, the myeloid lineage (e.g., neutrophils and their precursors), and the megakaryocytic lineage. In pregnancy, the plasma methylome may contain methylomic information from the fetus and the mother. The “cellular methylome” corresponds to the methylome determined from cells (e.g., blood cells) of the patient. The methylome of the blood cells is called the blood cell methylome (or blood methylome).

A “methylation profile” includes information related to DNA or RNA methylation for multiple sites or regions. Information related to DNA methylation can include, but not limited to, a methylation index of a CpG site, a methylation density (MD for short) of CpG sites in a region, a distribution of CpG sites over a contiguous region, a pattern or level of methylation for each individual CpG site within a region that contains more than one CpG site, and non-CpG methylation. In one embodiment, the methylation profile can include the pattern of methylation or non-methylation of more than one type of base (e.g., cytosine or adenine). A methylation profile of a substantial part of the genome can be considered equivalent to the methylome. “DNA methylation” in mammalian genomes typically refers to the addition of a methyl group to the 5′ carbon of cytosine residues (i.e., 5-methylcytosines) among CpG dinucleotides. DNA methylation may occur in cytosines in other contexts, for example CHG and CHH, where His adenine, cytosine or thymine. Cytosine methylation may also be in the form of 5-hydroxymethylcytosine. Non-cytosine methylation, such as N⁶-methyladenine, has also been reported.

A “methylation pattern” refers to the order of methylated and non-methylated bases. For example, the methylation pattern can be the order of methylated bases on a single DNA strand, a single double-stranded DNA molecule, or another type of nucleic acid molecule. As an example, three consecutive CpG sites may have any of the following methylation patterns: UUU, MMM, UMM, UMU, UUM, MUM, MUU, or MMU, where “U” indicates an unmethylated site and “M” indicates a methylated site.

The terms “hypermethylated” and “hypomethylated” may refer to the methylation density of a single DNA molecule as measured by its single molecule methylation level, e.g., the number of methylated bases or nucleotides within the molecule divided by the total number of methylatable bases or nucleotides within that molecule. A hypermethylated molecule is one in which the single molecule methylation level is at or above a threshold, which may be defined from application to application. The threshold may be 5%, 10%, 20%, 30%, 40%, 50%, 60%, 70%, 80%, 90%, or 95%. A hypomethylated molecule is one in which the single molecule methylation level is at or below a threshold, which may be defined from application to application, and which may change from application to application. The threshold may be 5%, 10%, 20%, 30%, 40%, 50%, 60%, 70%, 80%, 90%, or 95%.

The terms “hypermethylated” and “hypomethylated” may also refer to the methylation level of a population of DNA molecules as measured by the multiple molecule methylation levels of these molecules. A hypermethylated population of molecules is one in which the multiple molecule methylation level is at or above a threshold which may be defined from application to application, and which may change from application to application. The threshold may be 5%, 10%, 20%, 30%, 40%, 50%, 60%, 70%, 80%, 90%, or 95%. A hypomethylated population of molecules is one in which the multiple molecule methylation level is at or below a threshold which may be defined from application to application. The threshold may be 5%, 10%, 20%, 30%, 40%, 50%, 60%, 70%, 80%, 90%, and 95%. In one embodiment, the population of molecules may be aligned to one or more selected genomic regions. In one embodiment, the selected genomic region(s) may be related to a disease such as cancer, a genetic disorder, an imprinting disorder, a metabolic disorder, or a neurological disorder. The selected genomic region(s) can have a length of 50 nucleotides (nt), 100 nt, 200 nt, 300 nt, 500 nt, 1000 nt, 2 knt, 5 knt, 10 knt, 20 knt, 30 knt, 40 knt, 50 knt, 60 knt, 70 knt, 80 knt, 90 knt, 100 knt, 200 knt, 300 knt, 400 knt, 500 knt, or 1 Mnt.

The term “sequencing depth” refers to the number of times a locus is covered by a sequence read aligned to the locus. The locus could be as small as a nucleotide, or as large as a chromosome arm, or as large as the entire genome. Sequencing depth can be expressed as 50×, 100×, etc., where “x” refers to the number of times a locus is covered with a sequence read.

Sequencing depth can also be applied to multiple loci, or the whole genome, in which case x can refer to the mean number of times the loci or the haploid genome, or the whole genome, respectively, is sequenced. Ultra-deep sequencing can refer to at least 100× in sequencing depth.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1).

The terms “cutoff” and “threshold” refer to predetermined numbers used in an operation. For example, a cutoff size can refer to a size above which fragments are excluded. A threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. As another example, reference subjects with known classifications of one or more conditions and measured characteristic values (e.g., a methylation level, a statistical size value, or a count) can be used to determine reference levels to discriminate between the different conditions and/or classifications of a condition (e.g., whether the subject has the condition). A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The term “level of cancer” can refer to whether cancer exists (i.e., presence or absence), a stage of a cancer, a size of tumor, whether there is metastasis, the total tumor burden of the body, the cancer's response to treatment, and/or other measure of a severity of a cancer (e.g., recurrence of cancer). The level of cancer may be a number or other indicia, such as symbols, alphabet letters, and colors. The level may be zero. The level of cancer may also include premalignant or precancerous conditions (states). The level of cancer can be used in various ways. For example, screening can check if cancer is present in someone who is not previously known to have cancer. Assessment can investigate someone who has been diagnosed with cancer to monitor the progress of cancer over time, study the effectiveness of therapies or to determine the prognosis. In one embodiment, the prognosis can be expressed as the chance of a patient dying of cancer, or the chance of the cancer progressing after a specific duration or time, or the chance or extent of cancer metastasizing. Detection can mean ‘screening’ or can mean checking if someone, with suggestive features of cancer (e.g., symptoms or other positive tests), has cancer.

A “level of pathology” (or level of disorder) can refer to the amount, degree, or severity of pathology associated with an organism, where the level can be as described above for cancer. Another example of pathology is a rejection of a transplanted organ. Other example pathologies can include gene imprinting disorders, autoimmune attack (e.g., lupus nephritis damaging the kidney or multiple sclerosis), inflammatory diseases (e.g., hepatitis), fibrotic processes (e.g. cirrhosis), fatty infiltration (e.g., fatty liver diseases), degenerative processes (e.g. Alzheimer's disease), and ischemic tissue damage (e.g., myocardial infarction or stroke). A heathy state of a subject can be considered a classification of no pathology.

A “pregnancy-associated disorder” include any disorder characterized by abnormal relative expression levels of genes in maternal and/or fetal tissue. These disorders include, but are not limited to, preeclampsia, intrauterine growth restriction, invasive placentation, pre-term birth, hemolytic disease of the newborn, placental insufficiency, hydrops fetalis, fetal malformation, HELLP syndrome, systemic lupus erythematosus, and other immunological diseases of the mother.

The abbreviation “bp” refers to base pairs. In some instances, “bp” may be used to denote a length of a DNA fragment, even though the DNA fragment may be single stranded and does not include a base pair. In the context of single-stranded DNA, “bp” may be interpreted as providing the length in nucleotides.

The abbreviation “nt” refers to nucleotides. In some instances, “nt” may be used to denote a length of a single-stranded DNA in a base unit. Also, “nt” may be used to denote the relative positions such as upstream or downstream of the locus being analyzed. In some contexts concerning technological conceptualization, data presentation, processing and analysis, “nt” and “bp” may be used interchangeably.

The term “sequence context” can refer to the base compositions (A, C, G, or T) and the base orders in a stretch of DNA. Such a stretch of DNA could be surrounding a base that is subjected to or the target of base methylation analysis. For example, the sequence context can refer to bases upstream and/or downstream of a base that is subjected to base methylation analysis.

The term “kinetic features” or “kinetic signals” can refer to features derived from sequencing, including from single molecule, real-time sequencing. Such features can be used for base methylation analysis. Example kinetic features include upstream and downstream sequence context, strand information, interpulse duration, pulse widths, and pulse strength. In single molecule, real-time sequencing, one is continuously monitoring the effects of activities of a polymerase on a DNA template. Hence, measurements generated from such a sequencing can be regarded as kinetic features, e.g., nucleotide sequences.

A “machine learning model” (ML model) can refer to a software module configured to be run on one or more processors to provide a classification or numerical value of a property of one or more samples. An ML model can be generated using sample data (e.g., training data) to make predictions on test data. One example is an unsupervised learning model. Another example type of model is supervised learning that can be used with embodiments of the present disclosure. Example supervised learning models may include different approaches and algorithms including analytical learning, statistical models, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, nearest neighbor algorithm, probably approximately correct learning (PAC) learning, ripple down rules, a knowledge acquisition methodology, symbolic machine learning algorithms, subsymbolic machine learning algorithms, minimum complexity machines (MCM), random forests, ensembles of classifiers, ordinal classification, data pre-processing, handling imbalanced datasets, statistical relational learning, or Proaftn, a multicriteria classification algorithm. The model may include linear regression, logistic regression, deep recurrent neural network (e.g., long short term memory, LSTM), hidden Markov model (HMM), linear discriminant analysis (LDA), k-means clustering, density-based spatial clustering of applications with noise (DBSCAN), random forest algorithm, support vector machine (SVM), or any model described herein. Supervised learning models can be trained in various ways using various cost/loss functions that define the error from the known label (e.g., least squares and absolute difference from known classification) and various optimization techniques, e.g., using backpropagation, steepest descent, conjugate gradient, and Newton and quasi-Newton techniques.

The term “deep learning” may refer to artificial neural networks that use multiple layers in the network. The number of layers may be 2, 3, 4, 5, 6, 7, 8, 9, 10, 11, 12, 13, 14, 15, 20, 25, 30, 35, 40, or more, or any number in a range between and including these numbers.

The term “transformer” or “transformer layer” may refer to a machine learning model that adopts the mechanism of self-attention, differentially weighting the significance of each part of the input data. Transformers are designed to process sequential input data simultaneously rather than sequentially. Transformers are described in Vaswani et al., “Attention is All You Need,” arXiv: 1706.03762 (2017).

The term “real-time sequencing” may refer to a technique that involves data collection or monitoring during progress of a reaction involved in sequencing. For example, real-time sequencing may involve optical monitoring or filming the DNA polymerase incorporating a new base. As another example, real-time sequencing may involve electrical signal monitoring of ionic current through a nanopore when a nucleotide strand translocating that nanopore.

The term “electrical signal” may refer to a voltage or current that conveys information. The electrical signal could be expressed in a variety of regular and/or irregular signal waveform types and/or shapes such as square waves, rectangular waves, triangular waves, saw-toothed waveforms, or a variety of pulses and spikes. Electrical signal may include visual representations of variations of a voltage or current over time. The measurement of electrical signal could be sampled at particular times (e.g., millisecond). For example, the electrical current is sampled at a frequency of 1 kHz, 2 kHz, 3 kHz, 4 kHz, 5 kHz, 10 kHz, 20 kHz, 30 kHz, 40 kHz, 50 kHz, 100 kHz, etc.

The term “signal segment” or “segment” may refer to a portion of the trace of an

electrical signal associated with sequencing a particular nucleotide. The segment may correspond to the nucleotide determined from base-calling in nanopore sequencing. The segment may cover a certain duration of the trace. Different segments may have different durations. Segments may be non-overlapping. In some embodiments, the electrical signal amplitude may have a certain variation in the segment. For example, the electrical signal amplitude may be within 5%, 10%, 20%, 30%, or 40% of the mean or median electrical signal amplitude in the segment.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term “about” or “approximately” can mean within an order of magnitude, within 5-fold, and more preferably within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Standard abbreviations may be used, e.g., bp, base pair(s); kb, kilobase(s); pi, picoliter(s); s or sec, second(s); min, minute(s); h or hr, hour(s); aa, amino acid(s); nt, nucleotide(s); and the like.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the embodiments of the present disclosure, some potential and exemplary methods and materials may now be described.

### DETAILED DESCRIPTION

Accurate and efficient methods of detecting base methylations without enzymatic or chemical conversion (e.g., bisulfite conversion) are desired. Other methods and systems for directly detecting base methylation may depend on specific techniques (e.g., certain reagents or protocols) or equipment. Embodiments described herein use machine learning models to detect base methylations, which can be applied to a broad range of techniques or equipment. Embodiments may include machine learning models that capture local patterns (e.g., convolutional layers) and capture global patterns (e.g., transformer layers). Local patterns may be patterns resulting from elements in a given convolutional filter. Global patterns may be patterns resulting from outside a given convolutional filter, and the distance of these global patterns may be up to the size of a measurement window. Additionally, previous methods for detecting base methylations may not have been suited for detecting methylations at or near an end of a DNA fragment. Methods described herein can use signals from adaptors at the end of DNA fragments to help detect methylations.

The embodiments present in this disclosure can be used for DNA obtained from, but not limited to, cell lines, samples from an organism (e.g., solid organs, solid tissues, a sample obtained via endoscopy, blood, or plasma or serum or urine from a pregnant woman, chorionic villus biopsy, etc.), samples obtained from the environment (e.g., bacteria, cellular contaminants), food (e.g., meat). In some embodiments, the methods present in this disclosure can also be applied following a step in which a fraction of the genome is first enriched, e.g., using hybridization probes (Albert et al., 2007; Okou et al., 2007; Lee et al., 2011), or approaches based on physical separation (e.g., based on sizes, etc.) or following restriction enzyme digestion (e.g., MspI), or Cas9-based enrichment (Watson et al., 2019). While the invention does not require enzymatic or chemical conversion to work, in certain embodiments, such a conversion step can be included to further enhance the performance of the invention.

Embodiments of the present disclosure allow for improved accuracy or practicality or convenience in detecting base methylations or measuring methylation levels. The methylation may be detected directly. Embodiments may avoid enzymatic or chemical conversion, which may not preserve all methylation information for detection. Additionally, certain enzymatic or chemical conversions may not be compatible with certain types of methylations. Embodiments of the present disclosure may also avoid amplification by PCR, which may not transfer base-methylation information to the PCR products. Additionally, both strands of DNA may be sequenced together, thereby enabling the pairing of the sequence from one strand with its complementary sequence to the other strand. By contrast, PCR amplification splits the two strands of double-stranded DNA, so such pairing of sequences is difficult.

Methylation profiles, determined with or without enzymatic or chemical conversion, can be used for analyzing biological samples. In one embodiment, the methylation profiles can be used to detect the origin of cellular DNA (e.g., maternal or fetal, tissue, viral, or tumor). Detection of aberrant methylation profiles in tissues aid the identification of developmental disorders in individuals and the identification and prognostication of tumors or malignancies. Imbalances in methylation levels between haplotypes can be used to detect disorders, including cancer. Methylation patterns in a single molecule can identify chimeric (e.g., between a virus and human) and hybrid DNA, (e.g., between two genes normally unfused in a natural genome); or between two species (e.g., through genetic or genomic manipulation).

Methylation analysis may be improved by enhanced training, which may include narrowing the data used in a training set. Specific regions may be targeted for analysis. In embodiments, such targeting can involve an enzyme that either alone, or in combination with other reagent(s), may cleave a DNA sequence or a genome based on its sequence. In some embodiments the enzyme is a restriction enzyme that recognizes and cleaves a specific DNA sequence(s). In other embodiments, more than one restriction enzymes with different recognition sequences can be used in combination. In some embodiments, the restriction enzyme may cleave or not cleave based on the methylation status of the recognition sequences. In some embodiments, the enzyme is one within the CRISPR/Cas family. For example, genomic regions of interest can be targeted using a CRISPR/Cas9 system or other system based on guide RNA (i.e., short RNA sequences which bind to a complementary target DNA sequences and in the process guides an enzyme to act at a target genomic location). In some instances, methylation analysis may be possible without alignment to a reference genome.

### I. Machine Learning Techniques for Detecting Methylation

Embodiments described herein include a transformer layer in combination with convolutional layers to detect base methylations. The transformer layer uses attention mechanisms to encode data from results generated by one or more convolutional layers. A transformer is a deep learning model that uses the mechanism of self-attention, differentially weighting the importance or significance of each part of the input data at the current context.

Attention, analogous to cognitive attention, can enhance some parts of the input data while diminishing other parts such that the classification model can devote more focus to the small, but important, parts of the data. Learning which part of the data is more important than another part depends on the context and can be trained by gradient descent with the use of backpropagation process. The self-attention mechanism allows the inputs to interact with each other (“self”) and find out which parts of the data the model should pay more attention to (“attention”). Therefore, a model with attention may allow for fast learning and may achieve more accurate prediction by enhancing the influence of those more-relevant parts of input while reducing the influence of those less-relevant parts of the input.

One important process in a transformer is to transform the original input data matrix into three data matrices (i.e., query matrix (Q), key matrix (K), and value matrix (V)) by multiplying respective weight matrices, W_{Q}, Wₖ, and Wᵥ. The production between Q and K can be computed to form an attention score of each input part (also referred to as an attention filter, So). Then, multiplying attention filter (So) with the value matrix (V), namely S₀·V, to obtain the self-attention score (S), assigning high focus to the features that are more important to classification accuracy. One run of the above process is called one-head attention. One can apply multiple runs of such process to obtain different sets of relevant features which the model should pay differential attention to, called multi-head attention. We provide data showing machine learning models with the transformer layer were more accurate than models without the transformer layer.

A. Model framework

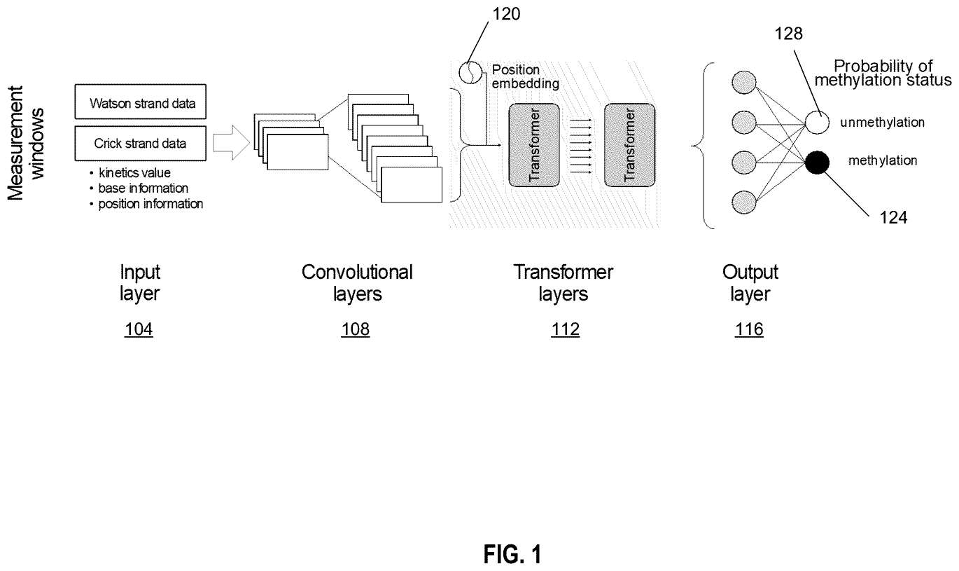

FIG. 1 shows an example model framework for DNA methylation using kinetic signals of a DNA polymerase during single molecule sequencing. Single molecule sequencing may include single molecule real-time (SMRT) sequencing or nanopore sequencing.

At stage 104, values of kinetic signals (“kinetics values” in FIG. 1) of single molecule real-time sequencing or other sequencing techniques and the corresponding identity of the bases (i.e., sequence context, shown as “base information” in FIG. 1) are organized into an input layer that could be a numeric matrix or vector. The “position information” refers to the relative position of a base on a strand. These kinetic signals may be any kinetic features described herein and are described in more detail elsewhere in this disclosure, including sections I.A.1 and II.A. Although stage 104 shows using both Watson strand data and Crick strand data, a single strand may be used instead of both strands.

At stage 108, the input layer is processed by one or more locally-connected layers (e.g., convolutional layers that share weights among a local group of sequence base positions). As an example, two one-dimensional (1D)-convolutional layers can be used. As a further example, 128 filters with a kernel size comprising all kinetic signals from 5 positions (i.e., five nucleotide positions in the measurement window,) are applied to each convolutional layer (also referred to as the latent dimension). Each position contributes two types of kinetic signals, namely IPD and PW. As the 1-D convolutional layer is used, all kinetic signals spanning rows from 5 positions will be automatically included. The kernel size in this example can be thus equivalent to 8×10. In some embodiments, the kernel size can include nucleotide positions of but not limited to 1, 2, 3, 4, 5, 6, 7, 8, 9, 10, 11, 12, 13, 14, 15, 16, 17, 18, 19, 20, 21, or more, or any combinations thereof. In some other embodiments, the number of filter(s) may be, but are not limited to at least 1, 2, 3, 4, 5, 6, 7, 8, 9, 10, 11, 12, 13, 14, 15, 16, 17, 18, 19, 20, 25, 30, 35, 40, 45, 50, 100, 200, 300, 400, 500, or combinations thereof. The number of filters may be within a range between and including any two of the numbers.

A batch normalization layer can be applied among convolutional layers with a rectified linear unit (ReLU) activation function. Batch normalization may normalize layers' inputs across the mini-batch by re-centering and re-scaling. The mini-batch refers to a subset of training samples. For example, batch normalization would process the values (denoted by x) in a matrix based on the overall mean (denoted by m) and standard deviation (denoted by sd) according to the formula (x-m)/sd. Therefore, the mean value and standard deviation of the input are close to 0 and 1, respectively. In examples, the output of one convolutional layer may be the input to a second convolutional layer. Each resultant output from convolutional layers may be integrated with positional information in a fragment of DNA being analyzed.

At stage 112, the convolutional results are added to the positional information (position embedding 120) as the input to the transformer layers. The transformer layers use the mechanism of self-attention to produce transformer results, which take into account global signal patterns.

At stage 116, the transformer results are processed by an output layer. The output layer processing can produce the probabilities of methylation (circle 124) and unmethylation (circle 128). An activation function can be used to provide the probabilities. Examples of activation functions include a softmax activation function, ridge activation functions (e.g., linear activation, ReLU activation, heaviside activation, logistic activation), radial activation functions (e.g., Gaussian, multiquadratics, inverse multiquadratics, polyharmonic splines), sigmoid, identity, and binary step. A methylation probability greater than a threshold value indicate a methylation.

The output layers at stage 116 include new data types used for determining methylation. FIG. 1 shows a method for generating these new data types. Methods may include specific machines for one or more stages of FIG. 1. These specific machines may include computers with specialized processors. For example, such processors may include a central processing unit (CPU) and a graphics processing unit (GPU) designed to accelerate computer graphics and image processing. In some embodiments, computers may include field programmable gate arrays (FPGAs) configured for machine learning detection of methylation.

In some embodiments, the probabilities of methylation may include probabilities of specific types of methylation. For example, the probabilities of methylation may include a probability of a 5 mC methylation, a probability of a 5 hmC methylation, a probability of a 6 mA methylation, or a probability of any other type of methylation. Circle 124 may be replaced with multiple circles for the multiple types of methylation.

### 1. Input Layers

In some embodiments, the activation functions may include, but are not limited to, binary step function, linear activation function, non-linear activation function, sigmoid/logistic activation function, hyperbolic tangent, exponential linear units (ELUs) function, Swish function, or Gaussian error linear units (GELUs). The output layer may include a number of neurons, where a number of arithmetic operations are performed. Such as multiplications by the weights and additions by biases. The number of neurons may be, but are not limited to, 1, 2, 3, 4, 5, 6, 8, 9, 10, 11, 12, 13, 14, 15, 16, 17, 18, 19, 20, 30, 40, 50, 100, 200, 300, 400, 500, or 1000. The number of neurons may be a range between and including any two of the numbers. The number of output layers could be but not limited to 1, 2, 3, 4, 5, 6, 7, 8, 9, 10, 20, 30, 40, 50, 100, etc. The number of output layers may be a number within a range between and including any two of the numbers. In some embodiments, one may use, but is not limited to, 2-D, 3-D convolutional layers, or other combinations.

Kinetic signals of a DNA polymerase may include the interpulse duration (IPD) and the pulse width (PW). IPD is a metric for the length of a time period between two emission pulses, each of which would be suggestive of a different incorporated fluorescently labeled nucleotide in a nascent strand. PW is another metric, reflecting polymerase kinetics, in association with the duration of the pulses related to a base incorporation. Kinetic signals corresponding to a series of sequenced nucleotides may be organized into a matrix (referred to as measurement window) based on relative sequence positions.

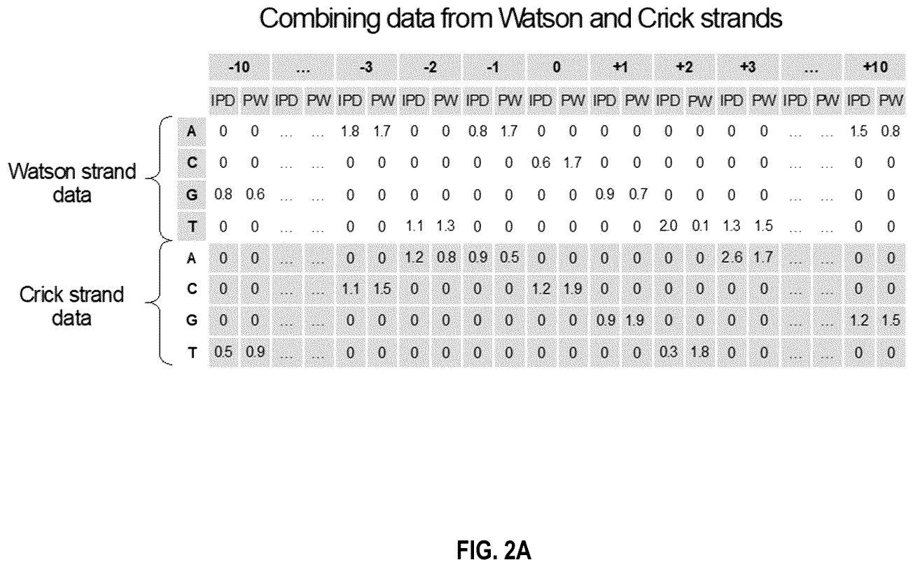

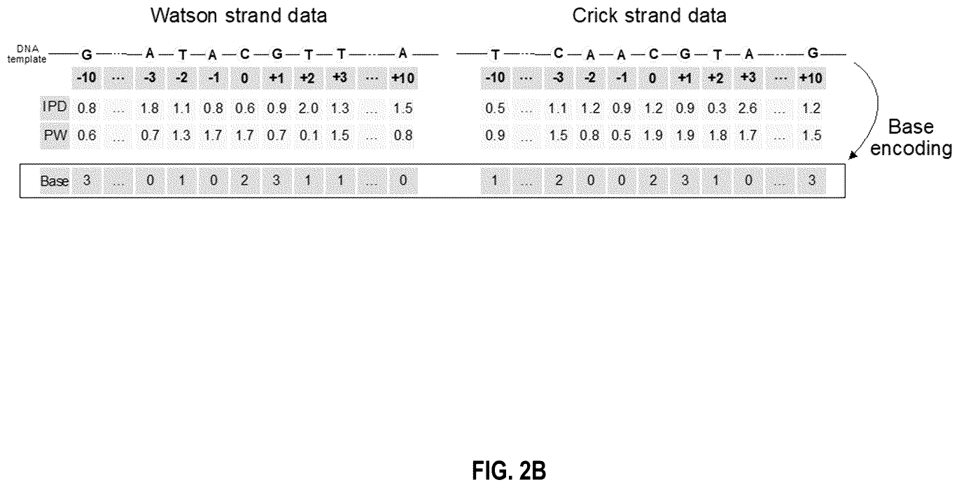

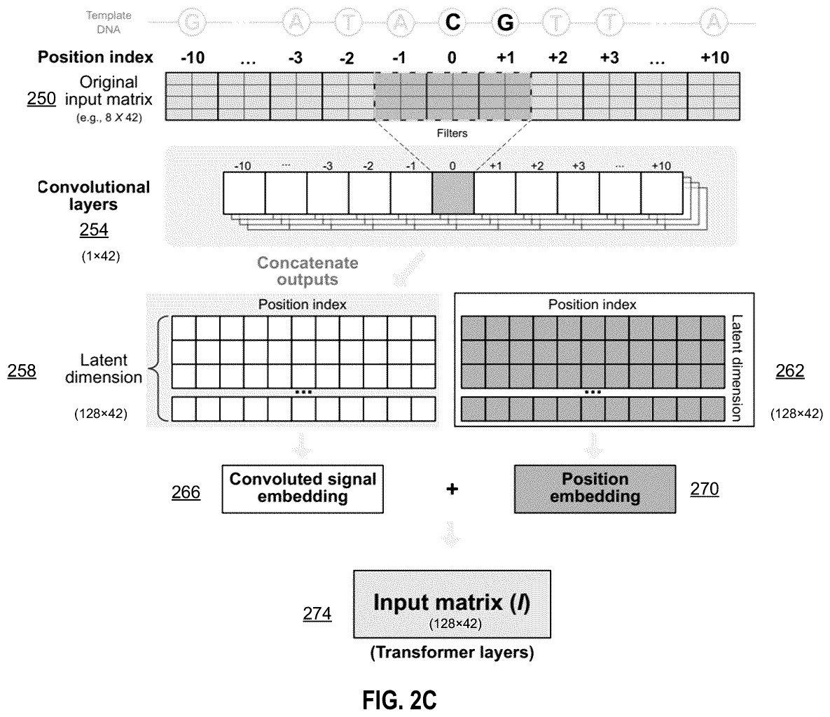

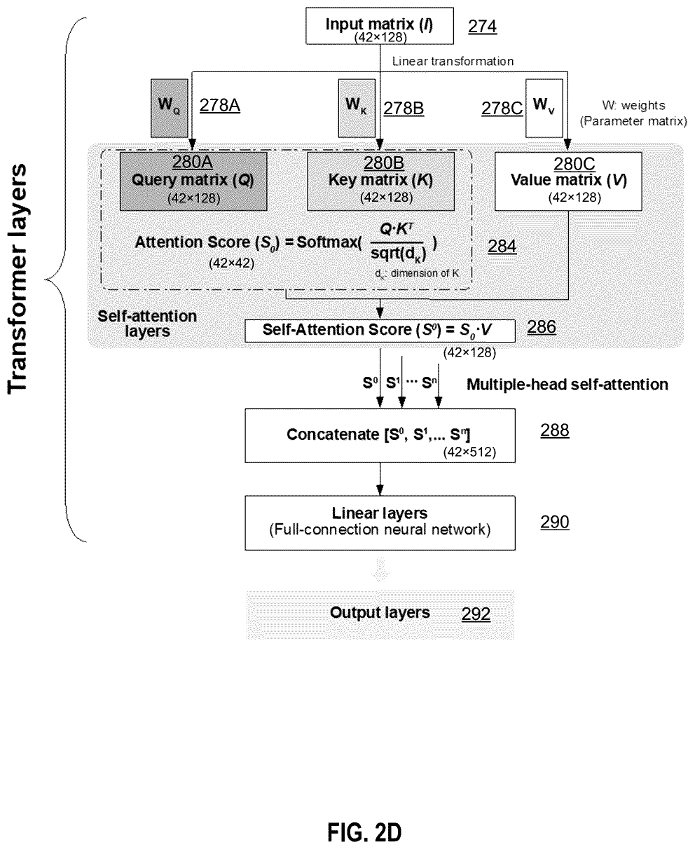

FIG. 2A shows an example measurement window combining Watson-strand data and Crick-strand data in a single 2-dimensional (2-D) data matrix. A measurement window may include kinetic signals including PW and IPD values originating from 10-nt upstream and 10-nt downstream of cytosine that is the target of methylation analysis. The first column of the matrix indicates the type of nucleotide that is studied. In the first row of the matrix, the position of 0 represented the target base for base methylation analysis. The relative positions of −1, −2, and −3 indicate the position 1-nt, 2-nt, and 3-nt, respectively, upstream of the base that was subjected to base methylation analysis. The relative positions of +1, +2, and +3 indicate the position 1-nt, 2-nt, and 3-nt, respectively, downstream of the base that was subjected to base methylation analysis. Each position includes two columns, which contain the corresponding IPD and PW values.

The four rows following the row with the IPD and PW headers correspond to four types of nucleotides (A, C, G, and T) in a strand (e.g., the Watson strand). The presence of IPD and PW values in the matrix depends on which corresponding nucleotide type was sequenced at a particular position. For example, as shown in FIG. 2A, at the relative position of 0, the IPD and PW values were shown in the row indicating “G” in the Watson strand, indicating that a guanine was called in the sequence result at that position. The other grids in a column that do not correspond to a sequenced base may be coded as “0”. As an example, as shown in FIG. 2A, the sequence information corresponding to the 2-dimensional (2-D) data matrix (FIG. 2A) is 5′-GATGACT-3′ for the Watson strand. A similar process of constructing the measurement window could be applied to data generated from the Crick strand.

The measurement windows may be used as an input layer for initializing the model training and testing. In some embodiments, the DNA lengths of the upstream of loci for base methylation analysis may be, but are not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 300, 400, 500, or 1000 nucleotides. In some embodiments, the DNA lengths of the downstream of loci for base methylation analysis could be but not limited to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 200, 300, 400, 500, 1000, etc. The lengths of the upstream and downstream of loci for base methylation analysis could be equal or not. The lengths may be a range between and including any two numbers disclosed.

In some embodiments, only one of the Watson strand or the Crick strand may be used. A model using single-stranded data as an input can detect methylation. Using only a single strand is discussed in more detail in section I.D.

FIG. 2B shows an example of a measurement window of data from one strand with base encoding. As shown in FIG. 2B, base information may be encoded by different digits: for example, A=0, T=1, C=2, and G=3. Such digitized base information may be organized as shown at the bottom of the 2-D data matrix. Data from the Watson strand and Crick strand may be used as two independent inputs. The data may then be processed with convolutional layers and transformer layers, as described with FIG. 1. In embodiments, the processed information matrices derived from the Watson and Crick strands may be concatenated vertically (e.g., as was done for FIG. 2A) or horizontally (as is shown in FIG. 2B) and further input into the output layer for producing the methylation probability.

The size of the measurement window may be, but is not limited to, 3 nt, 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 50 nt, 60 nt, 70 nt, 80 nt, 90 nt, 100 nt, 200 nt, 500 nt, or any range between and including any two of the numbers.

Some sequencing techniques (e.g., PacBio SMRT sequencing) involve ligating adaptor sequences to DNA fragments. Typically, the information related to the adaptor sequences is trimmed off such that the produced sequencing reads for end users are without adaptors. Hence, many CpG sites close to a 5′ end or a 3′ end of DNA sequence may not have sufficient flanking kinetic signals for methylation analysis. For example, for a size of measurement window of 21 centered on a cytosine, CpG sites within a distance of 10 nt to the 5′ end would not have sufficient flanking kinetic signals for methylation analysis. In embodiments, the kinetic signals from the trimmed adaptor sequences may be used and merges into the kinetic signals from DNA fragments of interest such that any CpG sites in sequenced DNA fragments, even if close to an end of the fragment, may be analyzed.

Measurement windows are described in US Patent Publication No. 2021/0047679 A1, filed Aug. 17, 2020, the entire contents of which are incorporated herein by reference for all purposes.

### 2. Dropout Layers

In some embodiments, the dropout layers may be applied between any layers mentioned in the stages to improve the model's generalizability and prevent overfitting. The dropout layers allow the ignoring of some of the features or neurons before being input to the next layer. The dropout layers may be implemented in a way that the input units for each layer are randomly set to 0 by a certain dropout rate (e.g., if one uses a dropout rate of 20%, 20% of neurons would be ignored and flagged as 0) at each step during training.

### 3. Convolutional Layers

A convolutional layer contains a set of filters (or kernels), the parameters of which are learned from training. The size of a filter is usually smaller than the actual size of the measurement window. Each filter slides across the input matrix horizontally and vertically and the dot product between the filter and the input is calculated at every spatial position, creating the convolutional output. For example, if a filter is 3×3, then the output may be a sum or average of values of the inputs and the output is assigned to the middle node. The convolutional process may be repeated with different filters. Such a process may capture the local patterns of neighboring signals (i.e., local signal patterns).

FIG. 2C illustrates one example process of integrating the results of convolutional layers and positional information of a DNA segment to generate an input matrix for downstream transformer layers. As shown in FIG. 2C, the original input matrix 250 may be a matrix with a dimension (i.e., shape) of 8×42 (e.g., FIG. 2A). Matrix 250 includes 8 rows indicating A, C, G, and T from each of the Watson and Crick stands and 42 columns indicating IP and PW values across positions in a 21-nt measurement window. Matrix 250 does not show all rows and columns to scale for clarity of the overall figure.

A filter kernel, e.g., with a dimension of 8×6 (i.e., all bases and 3 positions, including IP and PW for each position), can scan a measurement window from the left to right with a sliding step size (e.g., of 1). The process of convolutional layers may generate an output matrix with a dimension of 1×42 with paddings outside the edges (e.g., 8×5 zero padding matrix). Such a padding operation may allow the size of columns to be identical in both the input matrix and the output matrix. In some other embodiments, the filter kernel size could be nxm in which n may be, but is not limited to, 0, 1, 2, 3, 4, 5, 6, 7, 8, 9, 10, 11, 12, 13, 14, 15, 20, 25, 30, 35, 40, or any number in a range between and including these numbers, and m could be 0, 1, 2, 3, 4, 5, 6, 7, 8, 9, 10, 11, 12, 13, 14, 15, 20, 25, 30, 35, 40, or any number in a range between and including these numbers. The sliding step size may be 1, 2, 3, 4, 5, 6, 7, 8, 9, 10, 11, 12, 13, 14, 15, or any number in a range between and including these numbers. The filter kernel can have a filter kernel size that is less than a size of the window used to create the data structure.

Different filters may contain a set of different weights, which may generate different convolutional layers 254. One or more filters (e.g., with the same size) could be used and generate one or more output matrices in convolutional layers. The number of filters may be, but is not limited to, 1, 2, 3, 5, 6, 7, 8, 9, 10, 15, 20, 25, 30, 40, 50, or any number in a range between and including these numbers. As an example, there may be 128 convolutional filters.

All outputs from convolutional layers are concatenated and organized in a matrix 258 with a latent dimension based on the positional information in measurement window, named convolutional output. The latent dimension depends on the size of input matrix, the number of filters, and the size of each filter used (e.g., if padding is not performed). The convolutional results (e.g., convolutional output matrix 258) generated by the sliding filter kernel may be indexed by nucleotide positions in a measurement window. The positional indices may be stored in another matrix 262, representing the relative positional relationship. Matrix 258 containing convolutional output and matrix 262 containing positional indices are processed by embedding.

For example, if there are 128 convolutional filters, then matrix 258 and matrix 262 may have dimension 128×42.

The position index in matrix 262 can specify the position in a DNA segment relative to the CpG site in question. In this example, the relative positions are ranged from 10 nt upstream to 10 nt downstream of the CpG site.

Embedding refers to numeric operations regarding spatial transformations, for example, mapping an n-dimensional vector (or matrix) to an m-dimensional vector (or matrix), including linear transformations, rotations, and scaling. Embedding may increase or decrease the dimensionality, compared with the data matrix before embedding. Compared with traditional dimensionality reduction techniques such as principal component analysis (PCA), one important feature of embedding herein is that the embedding space (e.g., weights used in linear transformation) can be learnable according to the backpropagation and loss function. As a result, the addition of embedding layers may improve model performance.

As shown in FIG. 2C, convoluted signal embedding 266 may analyze and store data from convolutional layers.

### 4. Positional Indices

Position embedding 270 may store positional indices in a matrix of the same shape (dimensions) as the one used in the convoluted signal embedding 266. Relative position information may be encoded by periodic functions (e.g., sine, cosine, tangent, cotangent) as initial weights, and relative position information may be learnable during the training of the model.

Position embedding may incorporate information related to time series during sequencing to account for the order of the features. The position embedding aids the model in recognizing pattern information at any position in the feature map that may be derived from CNN process. Position embedding may facilitate the model in capturing the relationship of patterns between any positions in the feature map through self-attention mechanisms. Position embedding provides a space to contain the feature map, making the positions in the feature map trainable such that the pattern information between positions can be effectively learned.

Input matrix (I) 274 for input into transformer layers would include the results from Convoluted signal embedding and Positional embedding. In the original input matrix 250, the status in a column of a measurement window is a vector corresponding to a base position i. Input matrix (I) 274 is after the convolutional and embedding layers. In input matrix (I) 274, a column X can correspond to the base position i. Xis a vector with the convolutional outputs as the elements. Input matrix (I) may have the same dimension (e.g., 128×42) as matrix 258 and matrix 262.

Position encoding process by sine/cosine functions may refer to the following formulas for calculating positional information in latent dimension:

$$P {E}_{( p , 2 i )} = sin ( \frac{p}{1000 {0}^{2 i / l}} ) ,$$

$$and {PE}_{( p , 2 i + 1 )} = cos ( \frac{p}{1000 {0}^{2 i + 1 / l}} ) ,$$

where (p, 2i) indicates that a positional index, p, at the 2i^{-th} axis (an even axis), (p, 2i+1) indicates that a positional index, p, at the (2i+1)-th axis (an odd axis); l represents the size of latent-dimension (e.g., row number) of concatenated convolutional results.

### 5. Transformer Layers

One or more transformer layers can be applied to intermediate results, e.g., after a locally-connected layer is applied, such as one or more convolutional layers. As an example, two or three transformer layers can be applied after the convolutional neural network (CNN). A transformer layer (also referred to as a block in some contexts) can use the mechanism of self-attention allowing for differentially weighting the relevance of each part of the input data across all positions. Each transformer block can have a series of parameters for training, including the number of multiple-head self-attention, QKV biases (queries, keys, and values in attention mechanisms), and a ratio of multilayer perceptron. Multiple-head self-attention refers to a process by which attention mechanisms may be applied several times in parallel across the data input matrices. In one example, the number of multiple-head self-attentions was 4. In some embodiments, the number of multiple-head self-attentions may be 1, 2, 3, 5, 6, 7, 8, 9, 10, 15, 20, 30, 40, or 50, or any range between and including any two of the numbers.

In the transformer model, the attention mechanism is implemented using a number of numeric operations. The self-attention module takes n inputs and returns n outputs, allowing the inputs to interact with each other (i.e., “self”) to determine which inputs should be given higher weights (i.e., “attention”). The outputs are aggregates of these interactions and attention scores. In one embodiment, the numeric operations may be vectorized. In some context, the vector and matrix can be interconverted. For example, a matrix may be constructed from multiple vectors. As another example, a matrix can be indexed into different vectors.

As an example, the input vectors for the transformer layers in input matrix (I) 274 can be an intermediate result after operation of a layer, e.g., convolutional layers 254 as shown in FIG. 2C. As another example, a series of input vectors can correspond to the columns in a measurement window, e.g., as shown in FIG. 2A, without applying convolutional layers. Each column corresponds to the status of the nucleic acid at that position.

FIG. 2D shows how the input matrix (I) 274 is processed by a transformer layer. Input matrix (I) has a dimension, e.g., 42×128. Note that the matrix dimensions are swapped from FIG. 2C. Matrix 274 in FIG. 2D may be the transpose of matrix 274 in FIG. 2C. The input vectors in input matrix (I) 274 can interact with weight matrices (W_{Q}, W_{K}, and W_{V}) 278A, 278B, 278C that correspond to three representations, including key (K) matrix, query (Q) matrix, and a value (V) matrix. W_{Q}, W_{K}, and W_{V} exist for each input vector and can be viewed as parameters, e.g., weights of a neural network. Each weight vector can multiply a corresponding input vector to provide the representation vectors K, Q, and V (280B, 280A, 280C).

As to the values of these parameter matrices (K, Q, and V), one may randomly initialize weights (values) that are used in W_{Q}, W_{K}, and W_{V}. Each input matrix may be multiplied respectively by a set of weight matrices W_{Q}, W_{K}, and W_{V}, and each intermediate matrix result may be added to a corresponding bias matrix (B) to generate K, Q, and V. K 280B, Q 280A, and V 280C matrices may have the same dimensions as input matrix (I), e.g., 42×128.

For the input matrix corresponding to a base position I, Qᵢ can be used as the query to the key matrix of all the input matrix (including Ki and the other input matrix) for each of the positions. For example, an inner product can be applied between Qᵢ and the respective K matrix. The resulting values can be normalized on a scale between 0 and 1 (e.g., using an activation function). The normalized values can then multiply the value V matrix and be summed to obtain the Self-attention Score matrix.

Such multiplications may be conducted many times, such as, but not limited to, 1, 2, 3, 4, 5, 6, 7, 8, 9, 10, 15, 20, 25, 30, 35, 40, 45, 50, 100, 200, 300, 400, or 500 times, or any range between and including any two of the numbers. In embodiments, one may implement the transformer layer as below for a given input matrix (I) 274:
1. Preparing inputs (denoted by I) that are generated based on the related detail description of FIG. 2C, which is derived from convolutional layers and combined with positional information. Convolutional layers process the raw measurement windows having the kinetic signals including IPD and PW metrics, as well as identities of the bases.
2. Applying layer normalization for I, followed by the linear transformation into three matrices, named query matrix (Q), key matrix (K), and value matrix (V), respectively. For example, one may set Q=1·W_{Q}+B_{Q} where “·” represents the numeric operation of dot product; “W_{Q}” represents a weight matrix; and “B_{Q}” represents numeric biases that are added into such a linear transformation. Similarly, one may set K=I·W_{K}+B_{K} and V=I·W_{V}+B_{V}. Layer normalization normalizes each of the inputs in the batch across all features, which is different from batch normalization that normalizes each of the features across a batch.
3. Computing the attention score So (stage 284) for one process or run of self-attention layer according to an example formula in the following:

$${S}_{0} = Softmax ( Q \times {K}^{T} / \sqrt{{d}_{K}} ) ,$$

where (Q×K^{T}) represents the matrix multiplication between Q and K matrices in this practice. In another embodiment, one may use cosine similarity to calculate an attention score. Any similarity metric or projection between the two vectors can be used. D_{K} represents the dimension of matrix K (that is equal to the dimension of Q and V), and divided by the square root of dₖ represents a scale factor of the additive attention. The attention score S₀ is a matrix and is discussed in more detail below.
4. Computing one self-attention score (S) (stage 286) by multiplying the attention score with value matrix (V). One head of self-attention refers to one run of self-attention layers. Multiple-head of attention would generate several different self-attention scores (i.e., S⁰, S¹, . . . Sⁿ). In this situation, all self-attention scores may be concatenated to obtain a merged matrix (stage 288) as the intermediate output by self-attention process. The self-attention score is described in more detail below.
5. A set of full-connection neural network layers (stage 290) (possibly as well as Multilayer Perceptron [MLP] layer) to obtain the outputs (O) 292 of the transformer layers.
6. When repeating the above steps under the set of transformer layers, one may obtain outputs that can be used as downstream inputs for neuron network layers (e.g., fully-connected) generating the methylation probabilities.

Attention score Sois a matrix with a dimension of n×m, instead of a single value. In one example, n can be the measurement window size×2. If one uses 21-nt measurement window, each position has two kinetic features, namely IPD and PW. In example in FIG. 2D, n can be 42 (21×2). M can depend on the number of filter kernels, the filter kernel size, and paddings. For example, if one uses 128 filters with a filter kernel size of 8×6 that covers 3 nucleotide positions at each step of convolution, with the allowance of paddings, one can obtain an output during convolutional processes without any alterations in the column dimension [e.g., dimension of the output is (1×42) in convolutional layers 254]. The convolutional results can be concatenated such that one can obtain a 42×128 input matrix (I) for a transformer layer. When applying the embedding process to the input matrix (I) 274, the dimensionality of the input matrix 274 may not change. As shown in FIG. 2D, one can apply matrix transformation on input matrix (I) 274 using W_{Q}, W_{K}, and W_{V} matrices, obtaining Q, K, and V (280A, 280B, 280C) matrices, each with a dimension of 42×128. The softmax function is a function that turns a vector of K real values into a vector of real values that sum to 1. The formula is shown below:

$$softmax ( V ) = ( \frac{{e}^{{v}_{0}}}{{Σ}_{j = 1}^{k} {e}^{{v}_{j}}} , \frac{{e}^{{v}_{1}}}{{Σ}_{j = 1}^{k} {e}^{{v}_{j}}} , … , \frac{{e}^{{v}_{k}}}{{Σ}_{j = 1}^{k} {e}^{{v}_{j}}} ) ,$$

where V represents a vector containing k elements, e.g., V=(V₀, V₁, . . . , Vₖ). Therefore, the attention score (S₀) obtained from the matrix multiplication of Q and K^{T} is a matrix with a dimension of 42×42.

The self-attention score, S⁰, can be obtained by S₀·V, with a dimension of 42×128. If one performs 4 multi-head self-attention, the concatenation of multi-head self-attention results can lead to a 42×512 matrix, which may be further applied by a fully-connected layer.

In some embodiments, the convolutional results can be subjected to a pooling operation, such as but not limited to max pooling, averaging pooling, etc., instead of or in addition to concatenation. In FIG. 2D, if pooling were applied, then Q, K, and V matrices would be 42×1 each. At stage 286, S⁰ would have dimension 42×1 as well. With 4 multi-head self-attention, stage 288 would result in a merged matrix 42×4.

Layer normalization can be applied before and after each self-attention layer. The MLP layer at stage 290 may contain GELU layers to improve the performance. GELU represents Gaussian Error Linear Units, one type of activation functions. GELU can weight inputs by their value, rather than gate inputs as in Linear rectification function (ReLU). In one example, GELU can be defined as the below formula:

$$G E L U ( x ) = x \cdot \frac{1}{2} [ 1 + erf ( \frac{x}{\sqrt{2}} ) ] ,$$

where ‘erf’ is the error function defined by

$$erf ( z ) = \frac{2}{\sqrt{π}} {\int }_{0}^{z} {e}^{- {t}^{2}} d t .$$

The methods described herein integrate the convolutional network and transformer architectures. These methods may facilitate the modelling of the local signal patterns and global signal patterns, leading to the improvement in the methylation analysis.

### 6. Output Layers

An output layer with four neurons may be applied, with a softmax activation function to yield the probabilistic score for a CpG site of being methylated (i.e., probability of methylation), resulting in an output for the probability of methylation 124 and the probability of unmethylation (circle 128). In one embodiment, a sigmoid activation function can be used to yield the probabilistic score for a CpG site of being methylated (i.e., probability of methylation), resulting in an output for the probability of methylation (circle 124) and the probability of unmethylation (circle 128). In some embodiments, the probability of methylation (circle 124) may include several different probabilities, each probability for a different type of methylation. For example, a probability of a 5 mC methylation may be estimated. A probability for a 5 hmC methylation, a 6 mA methylation, or any other type of methylation may be estimated.

### B. Training and Testing Datasets

To demonstrate the effectiveness of the proposed model using convolutional neural network and transformer layers for methylation analysis (referred herein as the Enhanced Methylation Analysis [EMA] model or also referred to as HK model 2), we generated training datasets, including an unmethylated dataset and a methylated dataset. The unmethylated dataset contained sequencing results from amplified DNA that was prepared via whole genome amplification (WGA) (denoted as the WGA dataset). The use of unmodified nucleotides in the WGA resulted in the amplified DNA containing nearly no base methylations (with the exception of the small amount of input genomic DNA). The methylated dataset contained sequencing results from DNA treated by the M.SssI prior to sequencing (denoted as the M.SssI-treated dataset). M.SssI is a CpG methyltransferase, isolated from a strain of Escherichia coli that contains the methyltransferase gene from Sprioplasma sp. strain MQ1. M.SssI methyltransferase rendered CpG sites in a double-stranded DNA methylated (Greer et al., Cell. 2015;161:868-878). Among the sequenced CpG sites within the dataset of the M.SssI-treated sample, half was used for training a holistic kinetic (HK) model (Tse et al. Proc Natl Acad Sci USA. 2021 Feb. 2;118:e2019768118). The HK model does not include a transformer layer of the EMA model, among other differences. Within the WGA dataset, an equal number of CpG sites were randomly sampled for training the HK model. The remaining half of the CpG sites within the dataset of the M.SssI-treated sample and the same number from the WGA dataset were used for validation of the model. In this analysis, we used the Sequel II sequencing kit 2.0 on the PacBio Sequel II sequencer, obtaining WGA and M.SssI-treated DNA datasets for training and testing the HK model.

We used 2,000,000 CpG sites from an M.SssI-treated DNA sample (fully methylated) and 2,000,000 CpG sites from a WGA sample (fully unmethylated) to train the HK model. We used 2,000,000 CpG sites from an M.SssI-treated DNA sample (fully methylated) and 2,000,000 CpG sites from a WGA sample (fully unmethylated) to train the HK model EMA model. The optimal parameters of the HK or EMA model were obtained when the overall prediction error between the output scores calculated by the sigmoid function in the output layer, and desired target outputs (binary values: 0 or 1) reached a minimum by iteratively adjusting model parameters. The overall prediction error was measured by the sigmoid cross-entropy loss function in deep learning algorithms. The model parameters learned from the training datasets were used for analyzing the testing dataset to output a probabilistic score (referred to as the methylation score), indicating the likelihood of a CpG site being methylated. The methylation score output by the HK model was a continuous probabilistic score ranging from 0 to 1, instead of discrete binary values. For example, if the methylation score of a CpG site was 0.9, then applying a methylation score threshold of 0.5 would result in that CpG site being classified as methylated. In contrast, if the methylation score was 0.1, applying the same methylation score threshold of 0.5 would result in that site being classified as unmethylated.

### C. Accuracy Comparison Between HK Model and EMA Model

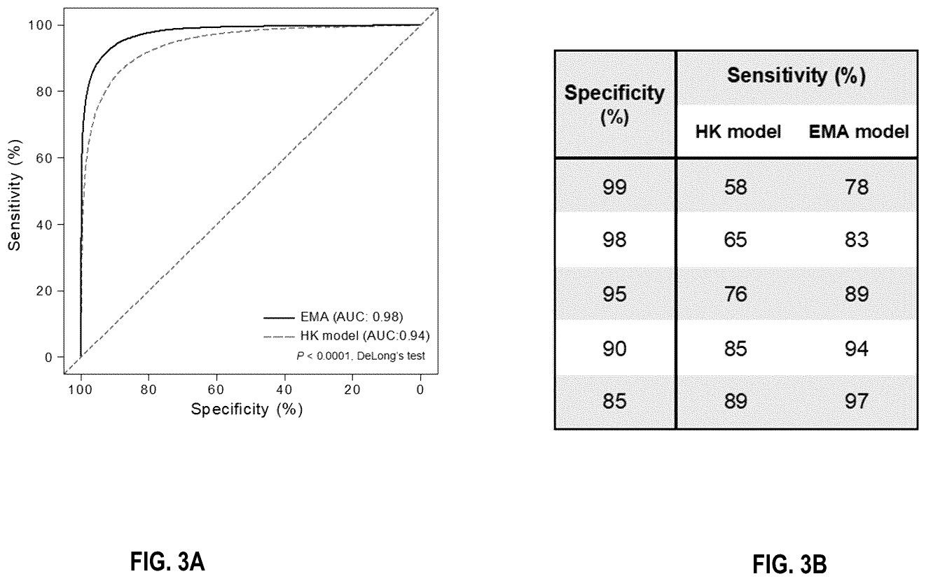

FIG. 3A shows receiver operating characteristic (ROC) curves comparing HK and EMA models. The x-axis shows specificity. The y-axis shows sensitivity. The dashed line shows results for the HK model. The solid line shows results for the EMA model. The area under the curve (AUC) of the EMA model was 0.98, which is improved over the HK model (AUC: 0.94) (P value<0.0001, DeLong's test).

FIG. 3B shows a table comparing sensitivities at given specificities for HK and EMA models. The first column lists different specificities. The second column lists the corresponding sensitivity of the HK model. The third column lists the corresponding sensitivity of the EMA model. At a specificity of 99%, the EMA model gave a sensitivity of 78%, which was better than the HK model's sensitivity of 58%. At a specificity of 95%, the EMA model gave a sensitivity of 89%, which was better than the HK model's sensitivity of 76%. At a specificity of 90%, the EMA model gave a sensitivity of 94%, which was better than the HK model's sensitivity of 85%.

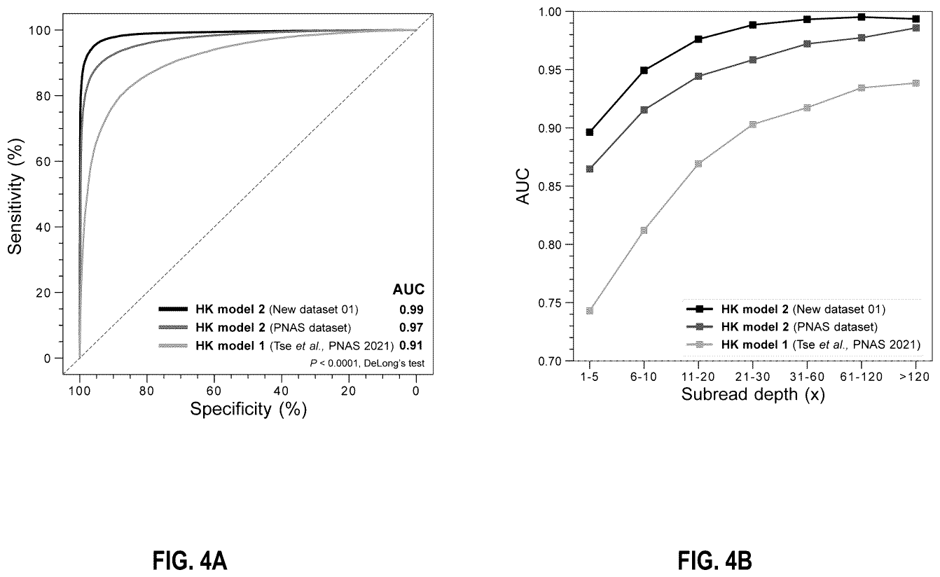

FIG. 4A shows receiver operating characteristic (ROC) curves for different models and datasets. The x-axis shows specificity. The y-axis shows sensitivity. The lowest curve shows HK model 1 from the previously published studyl. HK model 1 had an AUC of 0.91. The training dataset included PCR-amplified DNA (i.e., unmethylated DNA; the negative dataset) and M.SssI-treated DNA sets (i.e., methylated DNA; the positive dataset), each involving 0.35 million CpG sites.

The next lowest curve is labeled HK model 2, which used the same training dataset (referred to as PNAS dataset) as the previously published study (HK model 1)¹.)¹. The AUC was 0.97 with an independent testing dataset. HK model 2 was significantly improved, compared to HK model 1.

The highest curve is with HK model 2 with a different training dataset. The training dataset size was increased to 13 million CpG sites by preparing a new dataset (named New dataset 01) according to Tse et al.'s experimental protocols¹. The performance of HK model 2 was indeed further improved to an AUC of 0.99. If we defined a cutoff of base modification score of 0.5, we could obtain 96% specificity and 95% sensitivity.

FIG. 4B is a graph showing accuracy and subread depth. The x-axis is subread depth. The y-axis is AUC. The bottom curve shows HK model 1. The middle curve shows HK model 2 with PNAS dataset. The top curve shows HK model 2 with New dataset 01. A subread is defined as a read that begins at one adaptor sequence and ends at another adaptor sequence. In one embodiment, a subread that begins or ends in the middle of an insert sequence can be used. The subread depth is defined as the number of subreads obtained from a strand of a double-stranded DNA.

As shown in FIG. 4B, AUC values progressively increased for both HK model 2 and HK model 1, as the subread depth (x) increased. For example, the sensitivity and specificity in HK model 2 could reach 97% and 98% at a subread depth of >20× while the sensitivity and specificity were 87% and 89% at a subread depth of 5-10×). Of note, AUC values of HK model 2 trained by a large training dataset size (New dataset 01) showed consistent improvement across different subread depths, suggesting the robustness of HK model 2. There was a 22% increase in AUC for low subread depths (1-5×) and a 6% increase for high subread depths (>120×), when comparing the HK model 2 (New dataset 01) to HK model 1¹.1¹.

### D. Single-Stranded Model

The data for HK model 2 shown above involved combined Watson and Crick data (i.e., double-stranded HK model 2). We explored the performance of HK model 2 when analyzing single-stranded data. Analyzing the strand-specific methylation patterns would broaden the applicability of the model proposed in this study. For example, the strand-specific HK model makes it possible to dissect DNA hemi-methylation which was reported to occur at CTCF (CCCTC-binding factor)/cohesin binding sites and play a role in driving chromatin assembly¹³.

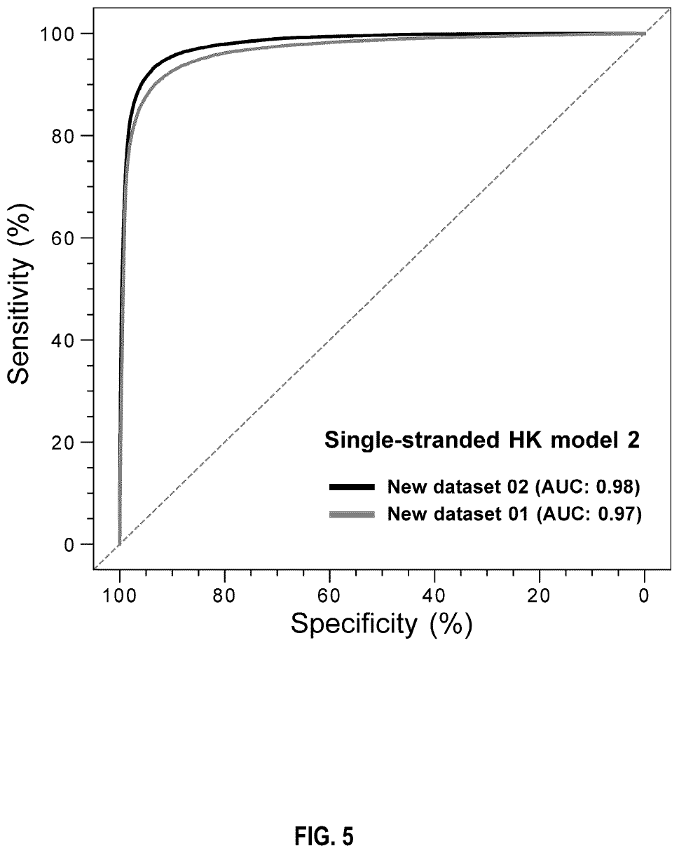

FIG. 5 is an ROC curve for HK model 2 with using only single strands. The x-axis is specificity. The y-axis is sensitivity. FIG. 5 shows that the single-stranded HK model 2 could still achieve an AUC of 0.97 using New dataset 01, without obviously deteriorating the model performance compared to double-stranded HK model 2. FIG. 5 also shows single-stranded HK model 2 for New dataset 02, which has a higher AUC of 0.98. New dataset 02 was prepared with a different protocol than New dataset 01. The protocol is described below.

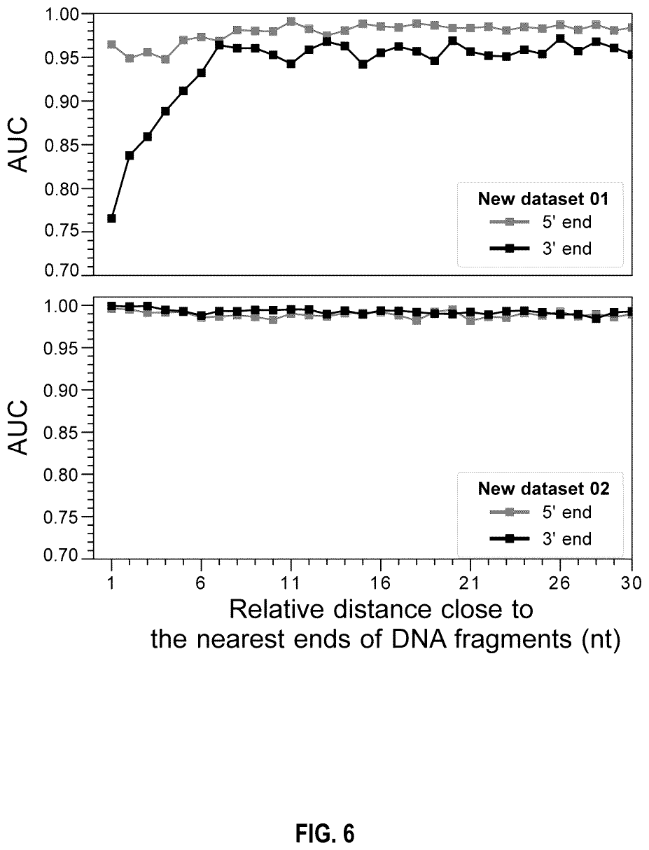

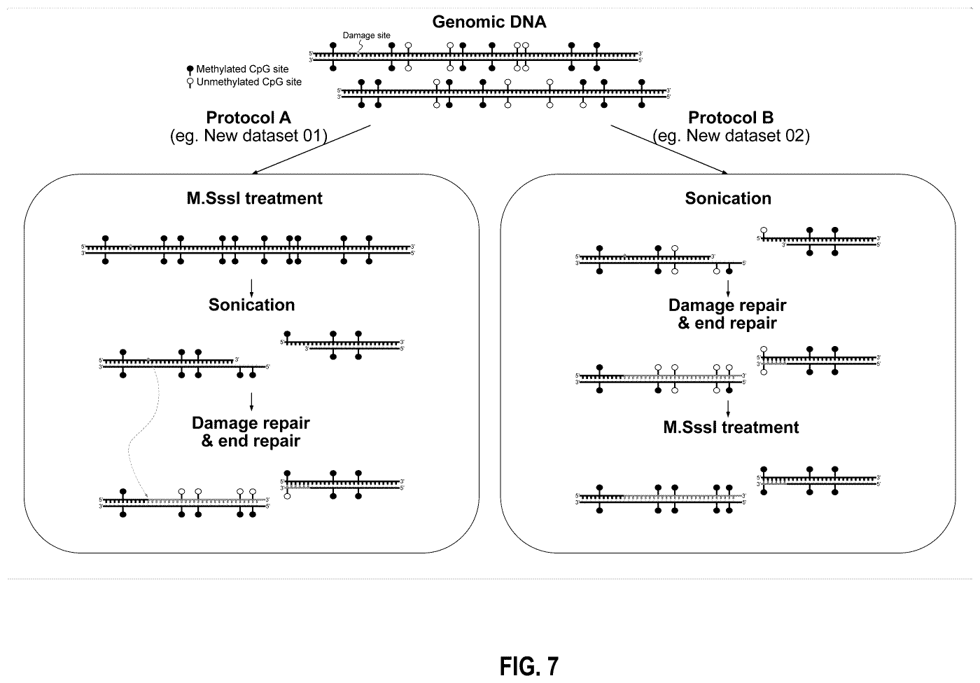

FIG. 6 shows the AUC of methylation analysis for CpG sites at positions relative to the nearest end of sequenced fragments for two datasets. The x-axis shows the relative distance to the nearest ends of DNA fragments (nt). The y-axis shows the AUC. The top graph shows results from New dataset 01 (by protocol A). The bottom graph shows results from New dataset 02 (by protocol B).

As seen in the top graph of FIG. 6, the performance of analyzing CpGs proximal to the 3′ end was generally worse than those proximal to the 5′ end. The closer to the 3′ end, the lower the AUC value would be. We hypothesized that the decreased performance of the single-stranded model for those CpG sites close to the 3′ end might be the unmethylated cytosines, which were introduced into those M.SssI-treated DNA molecules containing jagged ends during the DNA end repair. To confirm such a possibility, we revised Protocol A to Protocol B in which the end repair step was performed before the step of M.SssI treatment to generate another new training dataset, namely New dataset 02.

The use of New dataset 02 enabled the differentiation between methylated and unmethylated cytosines with an AUC of 0.98, confirming that the new protocol B was valid. More importantly, the discrepancy of AUC between the proximal regions of 5′ and 3′ ends shown in using single-stranded HK model 2 disappeared, as seen in FIG. 6.

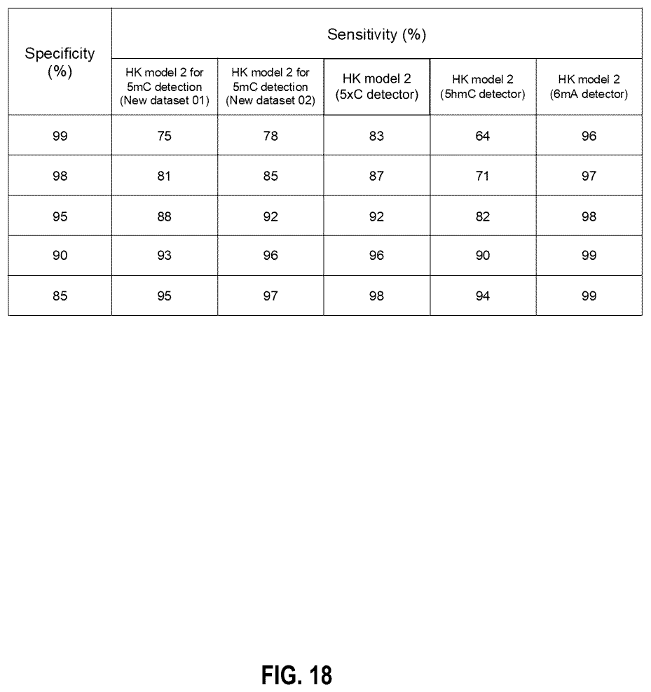

FIG. 18 illustrates the protocol for improved performance of the single-stranded model for methylation sites close to the 3′ end. An open lollipop indicates an unmethylated CpG site. A filled-in lollipop indicates a methylated CpG site. Protocol A, resulting in New dataset 01, is shown on the left branch. Protocol B, resulting in New dataset 02, is shown on the right branch.

Protocol A starts with M.SssI treatment, which methylates all the CpG sites. The genomic DNA is then sonicated. Damage repair and end repair is then performed. However, this damage repair and end repair result in unmethylated CpG sites being added to the fragments.

Protocol B starts with sonication. Damage repair and end repair is then performed. Once again, unmethylated CpG sites are added to the fragments. The M.SssI treatment is then performed, which methylates the unmethylated CpG sites. As a result, all CpG sites are methylated. Protocol B can be used to generate training sets for single-stranded models.

### E. Differentiating Between Different Methylation Types

Methods described herein can not only detect the presence of a methylation but also differentiate between different types of methylations. Such methods may involve a single model to determine the type of methylation present rather than using multiple models to test whether each particular type of methylation is present. The detection of different types of methylation may use a single-stranded model or a double-stranded model.

### 1. Differentiating Between 5 mC and 5 hmC With Testing Dataset

A training dataset that includes 5 hmC modifications was prepared. In contrast to the preparation of 5 mC modifications at CpG sites using a single methyltransferase (M.SssI), there is currently no such methyltransferase whose end product of enzymatic reaction will be 5 hmC. TET (ten-eleven translocation) proteins can catalyze the stepwise oxidation of 5 mC to produce a combination of 5-hydroxymethylcytosine (5 hmC), 5-formylcytosine (5 fC), and 5-carboxylcytosine. The proportions of these oxidized cytosines in a TET-treated DNA mixture vary depending on the incubation time.

It was reported that DNA treated by TET2 for approximal 5 minutes possibly led to a large percentage of 5 mC and 5 hmC present in the reaction product, with a relatively small contribution of 5 fC and 5 caC¹⁴. Hence, we used TET to treat the DNA previously methylated by M.SssI for 5 minutes, obtaining the training dataset approximating the mixture of 5 mC and 5 hmC modifications (named TET-5 xC dataset).

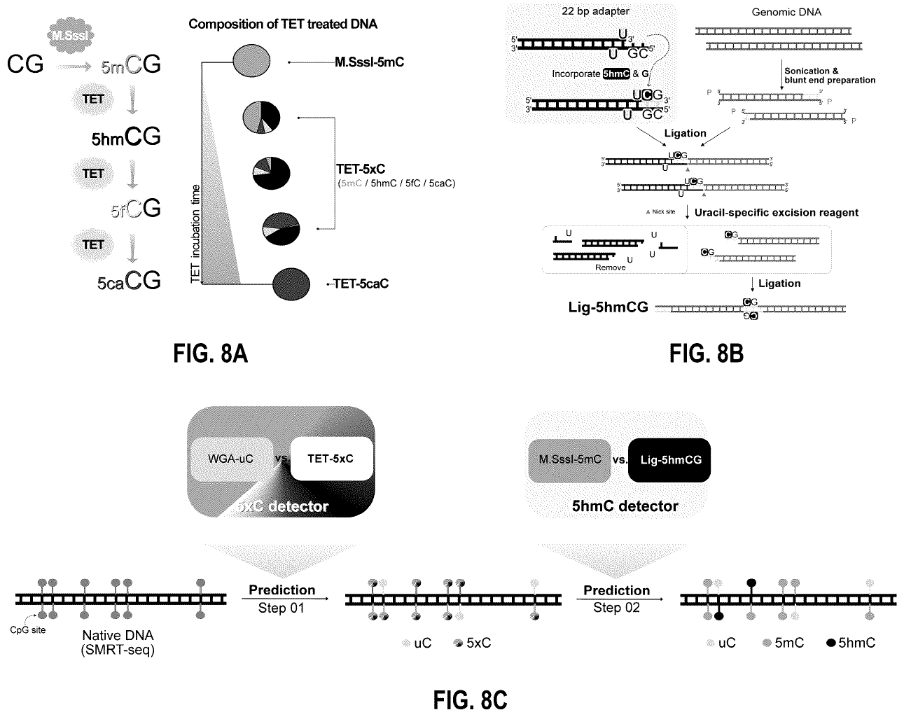

FIG. 8A shows the composition of TET-treated DNA. The amount of 5 hmC, 5 fC, and 5 acC varies with incubation time.

FIG. 8B shows the preparation of the 5 hmC detection dataset (named Lig-5 hmCG) using ligation.

FIG. 8C shows the analytical workflow for 5 mC and 5 hmC detection. Based on TET-5 xC and WGA-uC datasets, we established a model for determining the 5 xC and uC modifications (5xC detector). Based on M.SssI-mC and Lig-5 hmCG, we established a model for further resolving 5 xC into 5 mC and 5 hmC modifications (5 hmC detector).

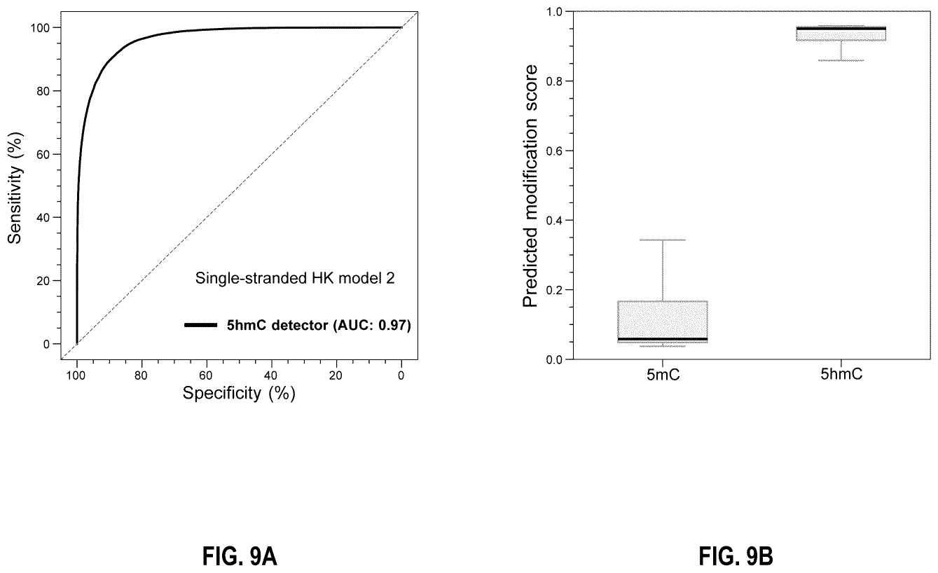

FIG. 9A shows an ROC curve of the testing datasets for the 5 xC and 5 hmC detectors. Sensitivity is shown on the y-axis. Specificity is shown on the x-axis. We analyzed a total of 18,040,000 CpG sites and 325,851 CpG sites for 5 xC and 5 hmC detectors, obtaining AUC values of 0.99 and 0.97, respectively. Using a base modification score cutoff of 0.5, 93% specificity and 94% sensitivity were obtained for differentiating between uC and 5 mC, while 85% specificity and 94% sensitivity were between 5 hmC and 5 mC.

FIG. 9B shows box plots of the modification scores predicted by the 5 hmC detector in the testing dataset. The x-axis shows either 5 mC or 5 hmC modification. The y-axis shows the predicted modification score. The modification scores of 5 hmC (median: 0.95; IQR: 0.92-0.96) were much higher than that of 5 mC (median: 0.06; IQR: 0.05-0.17) (P value<0.0001, Mann-Whitney U test).

FIG. 9A and FIG. 9B demonstrate that models can differentiate accurately between 5 mC and 5 hmC modifications.

### 2. Differentiating Between 5 mC and 5 hmC in Biological Samples

We further used biological samples to demonstrate the validity of 5 hmC detector. A buffy coat DNA sample was obtained from a healthy individual, and commercial brain DNA samples were obtained through EpigenTek. We used bisulfite sequencing (BS-seq) and Tet-assistant bisulfite sequencing (TAB-seq) to deduce the 5 xC (approximately the total level of 5 mC and 5 hmC) and 5 hmC levels in the buffy coat and brain samples, with a sequencing depth of haploid genome of at least around six folds.

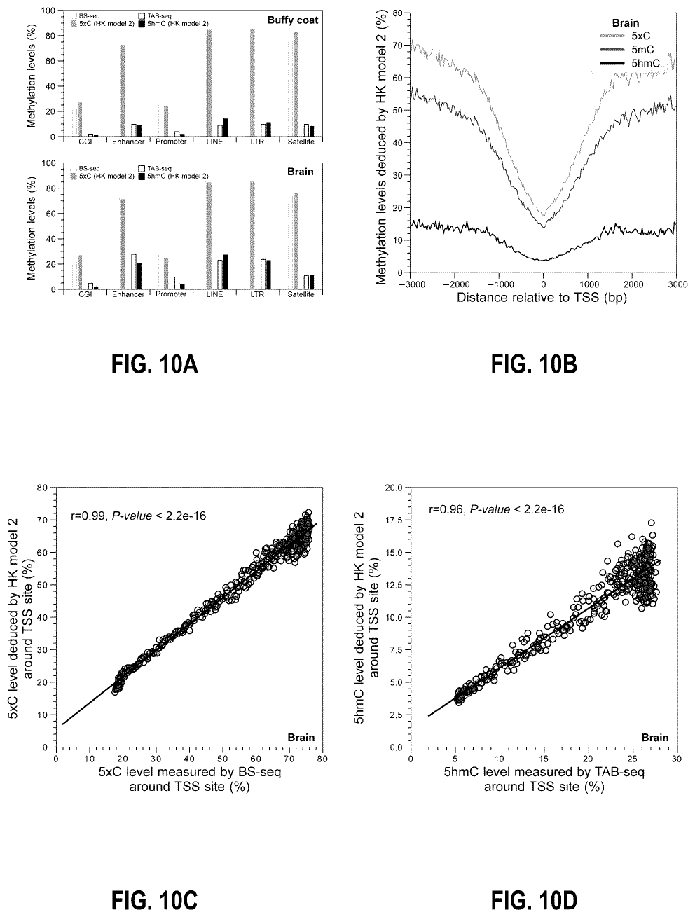

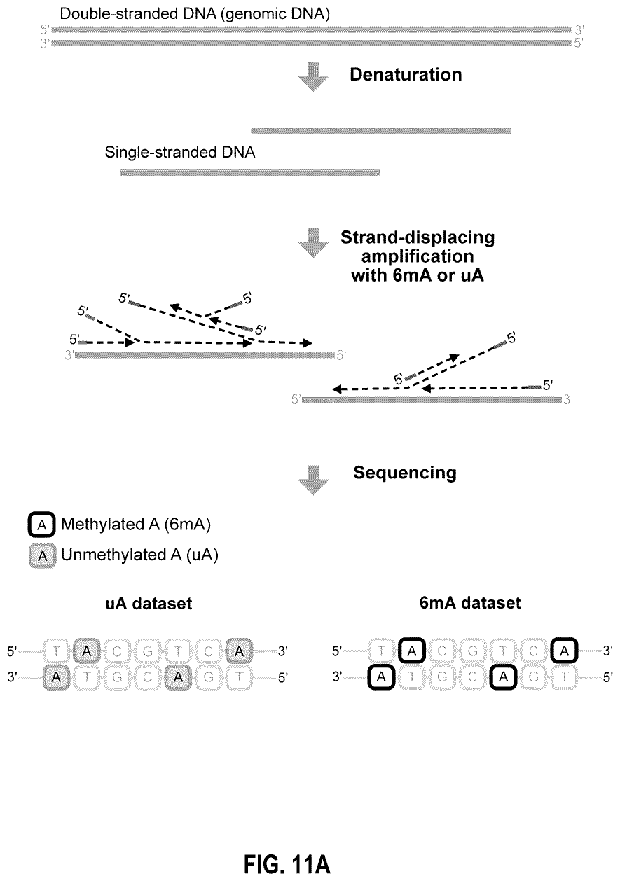

FIG. 10A shows methylation levels measured by different approaches in buffy coat and brain samples across different genomic regions of interest. The y-axis shows the methylation levels. The x-axis shows the genomic regions of interest. CGI is CpG island. LINE is long interspersed nuclear element. LTR is long terminal repeat. The top graph shows buffy coat. The bottom graph shows brain samples.

FIG. 10A shows that the 5 hmC modifications deduced by HK model 2 were found to be enriched in the brain across CpG islands (CGIs), enhancers, promoters, and repeat regions (e.g., LINE, LTR, and Satellite) with levels ranging from 2.23% to 27.47%, compared with the buffy coat sample (range: 1.19-14.33%). Such 5 hmC patterns were in great agreement with the data shown in TAB-seq results [Range of 5 hmC level: 4.78-27.64% (brain) versus 2.04-9.78% (buffy coat)]. The total levels of cytosine modification were found to be generally consistent between the measurements of HK model 2 (indicated by 5 xC) and BS-seq in both the buffy coat and brain.

FIG. 10B shows methylation levels predicted by HK model 2 in human brain samples around transcription start sites (TSS) sites. The x-axis shows distance (bp) relative to TSS sites. The y-axis shows methylation levels deduced by HK model 2. The top line is 5 xC methylation. The middle line is 5 mC methylation. The bottom line is 5 hmC methylation.

Notably, a sharp dip of both 5hmC and 5 mC levels surrounding TSS was clearly seen in the results of the brain deduced by HK model 2, with the levels of 5 hmC consistently lower than that of 5 mC. Such patterns were greatly in line with the previous reports7.

FIG. 10C shows the correlation of the 5 xC levels in brain samples measured by the HK model 2 and BS-seq. The x-axis shows the 5 xC level (%) measured by BS-seq around the TSS site. The y-axis shows the 5 xC level (%) deduced by HK model 2 around TSS site.

FIG. 10D shows the correlation of the 5 hmC levels (%) in brain samples measured by the HK model 2 and TAB-seq. The x-axis shows the 5hmC level (%) measured by TAB-seq around the TSS site. The y-axis shows the 5 hmC level (%) deduced by HK model 2 around TSS site.

Importantly, the 5 xC and 5 hmC levels analyzed by HK model 2 across positions nearby TSS were linearly correlated with those measured by BS-seq (Pearson's r: 0.99; P value<0.0001) and TAB-seq (Pearson's r: 0.96; P value<0.0001). The 5 mC methylation can be determined by 5 xC methylations that are not determined to be 5 hmC. These results demonstrate that models can differentiate between 5 mC and 5 hmC methylations.

### 3. Detection of 6 mA and 4 mC

Some DNA modifications showed a much sharper kinetic alterations at the modified sites, such as 6 mA or 4 mC, compared with 5 mC (Flusberg B A et al., Nat methods 2010; Clark T A et al., Nucleic Acids Res 2012). A previous method of detecting those DNA modifications, such as 6 mA, involved comparing the IPD values at adenine (A) sites from native DNA sequencing data with control IPD values from either methylation-free whole-genome amplified DNA or precomputed in silico IPD models¹⁵. It was reported that the fixed cutoff of IPD ratio for 6 mA detection would introduce false positive calls, especially from genomic regions with high sequencing depth16. These false positive calls and general accuracy issues are addressed with embodiments described herein. Using HK model 2 framework, we developed a new adaptive method, including producing the specific training dataset and correcting kinetic patterns in a measurement window, to achieve a high performance of both 6 mA and 4 mC detection.

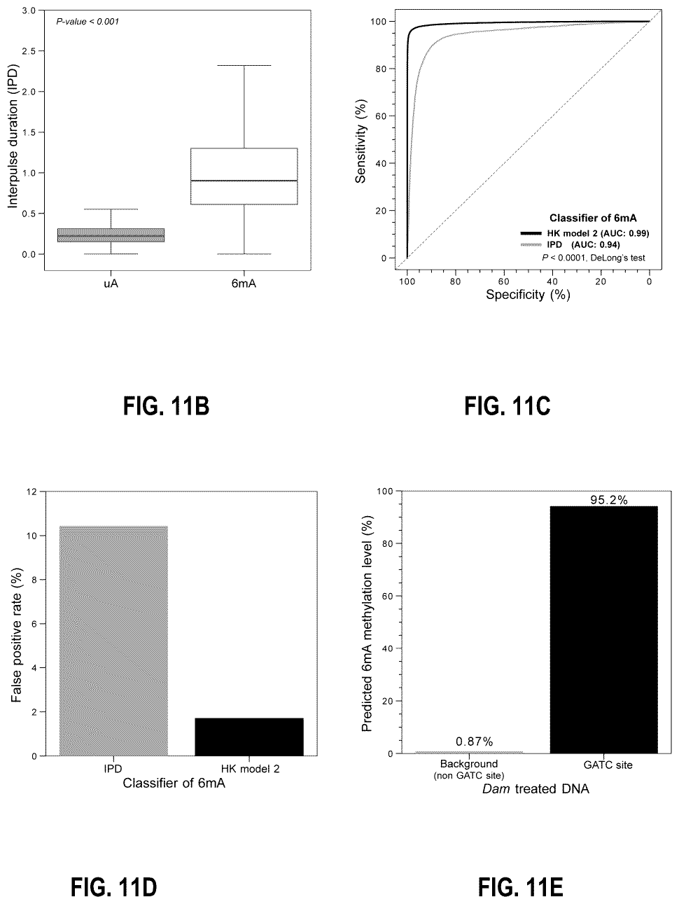

FIG. 11A is a schematic for preparing the unmethylated and methylated adenine datasets (i.e., uA and 6 mA datasets). We applied the whole-genome amplification with the presence of 6 mdATP such that nearly all adenine sites in amplified DNA molecules would be 6 mA (named WGA-6 mA dataset). The corresponding negative dataset could be obtained from the whole-genome amplification with unmodified dNTP (named WGA-uA dataset). A similar training dataset was generated for 4 mC.

FIG. 11B shows the IPD distributions in uA and 6 mA datasets. The y-axis shows the IPD. The x-axis shows the adenine methylation status. The IPD values on 6 mA site were significantly higher than those on uA sites (median: 0.90 versus 0.22; P value<0.0001), suggesting the successful introduction of 6 mA to the amplified DNA.

FIG. 11C shows ROC curves of 6 mA detection based on HK model 2 and only the IPD metric. The x-axis shows specificity. The y-axis shows sensitivity. The 6 mA detector has an AUC of 0.99, which was superior to the analysis based on IPD values of A sites (AUC: 0.94).

FIG. 11D shows false positive rates of 6 mA detection based on HK model 2 and only the IPD metric. The x-axis shows the classifier of 6 mA. The y-axis shows the false positive rate. If a cutoff of 6 mA modification score was set as 0.5, the sensitivity and specificity were 96% and 98%, respectively. The corresponding false positive rate of HK model 2 was 1.7%, which was greatly lower than the method based on IPD metric only (10.4%).

FIG. 11E shows 6 mA methylation levels determined by HK model 2 in non-GATC and GATC contexts in the Dam-treated DNA sample. The x-axis shows the type of site (non-GATC and GATC). The y-axis shows predicted 6 mA methylation level. We further validated the performance of the 6 mA detector using DNA molecules that had been treated by the E. coli DNA adenine methyltransferase enzyme (Dam), which is known to efficiently add a methyl group to the adenine (i.e., 6 mA) at the sequence context of 5′-GATC-3′. FIG. 11E shows that 95.2% of GATC motifs were determined to have 6 mA modifications, whereas only 0.87% of adenine sites within non-GATC contexts had 6 mA modifications. The result further confirmed the validity of 6 mA determination using the 6 mA detector. In some examples, based on the position to which the methyl group is transferred, DNA methyltransferases can be divided into two classes: exocyclic amino methyltransferases and endocyclic methyltransferases. Exocyclic amino methyltransferase transfers a methyl group to the N4 position of cytosine (4 mC) or the N6 position of adenine (6 mA), e.g., Dam and CcrM. Endocyclic methyltransferase methylates cytosine at the C5 position (5 mC), e.g., Dcm (Wion et al. Nat. Rev. Microbiol. 2006;4:183-192; Kumar et al. Nucleic Acids Res. 2018;46:3429-3445; Chen et al. Nat Commun. 2022; 13:1248). These variants may be included in the analysis based on HK model 2.

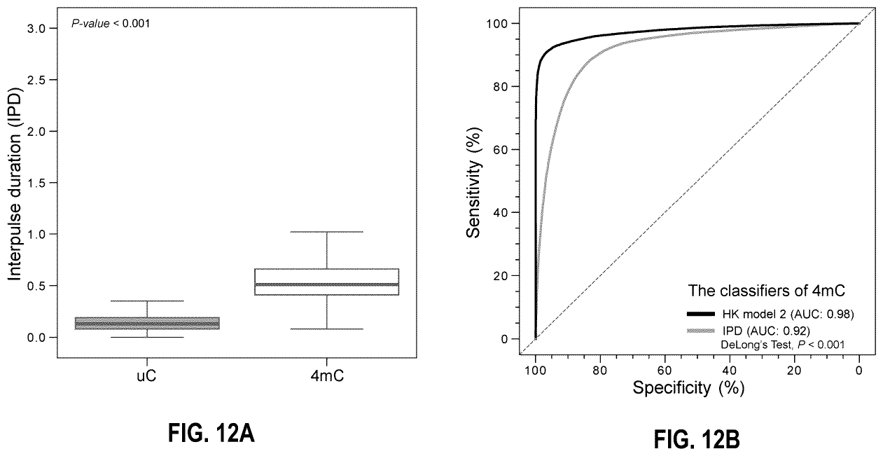

FIG. 12A shows the IPD distributions in uC and 4 mC datasets. The y-axis shows the IPD. The x-axis shows the cytosine methylation status. The IPD values on 4 mC site were significantly higher than those on uC sites (median: 0.54 versus 0.18; P value<0.001), suggesting the successful introduction of 4 mC to the amplified DNA. The increase of IPD associated with 4 mC appeared to be lower than 6 mA (median: 0.54 versus 0.90).

FIG. 12B shows ROC curves of 4 mC detection based on HK model 2 and only the IPD metric. The x-axis shows specificity. The y-axis shows sensitivity. The 4 mC detector has an AUC of 0.98, which was superior to the analysis based on IPD values of C sites (AUC: 0.92). The AUC of classification of 6 mA from uA is 0.94 whereas the AUC of classification of 4 mC from uC is 0.92, suggesting that the classification of 4 mC directly using IPD values would be more challenging.

To evaluate the performance of genome-wide 6 mA detection in biological samples, we applied HK model 2 to analyze microbial DNA (with an average of 220-fold coverage). It was known that the sequence motif GATC was characterized with 6 mA modifications in E. coli and S. enterica but not in B. subtills, E. faecalis, L. mono, and S. aureus^{17,18}. 6 mA methylation levels at GATC across various microbes were analyzed by HK model 2.

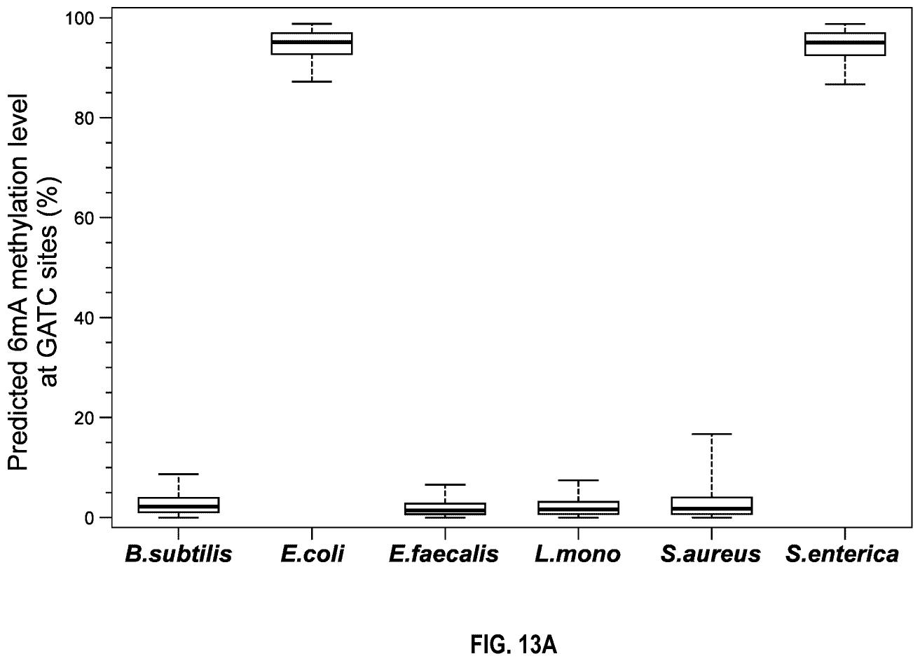

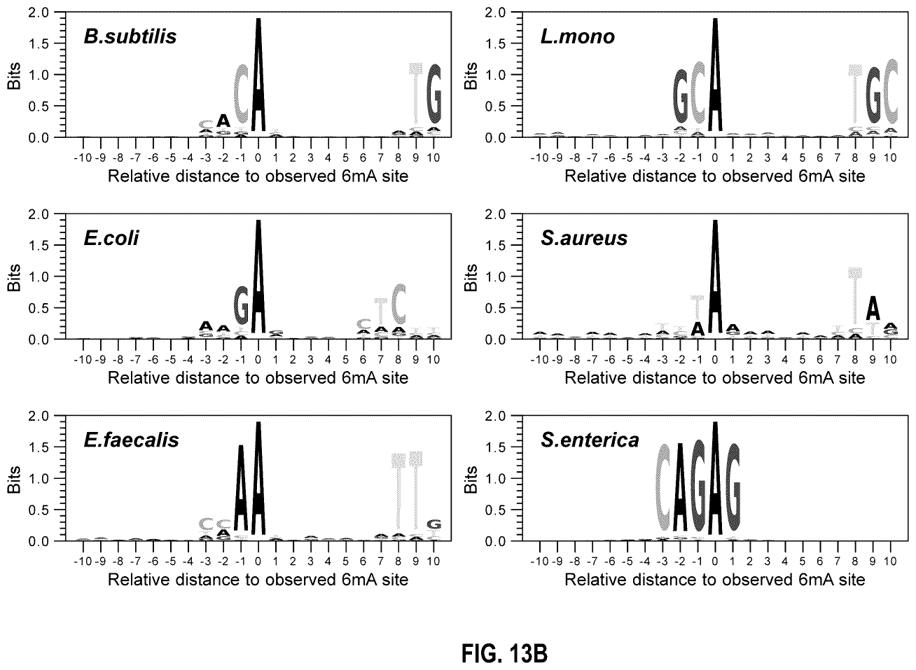

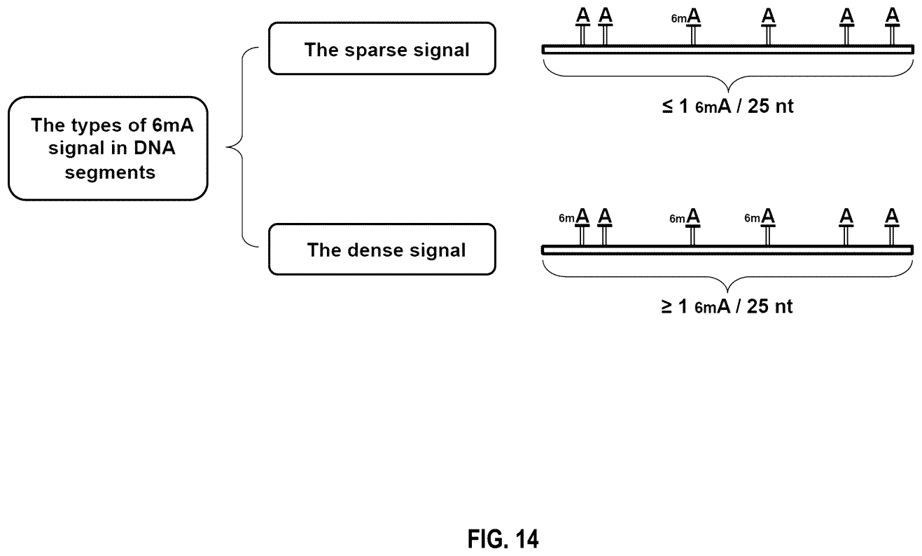

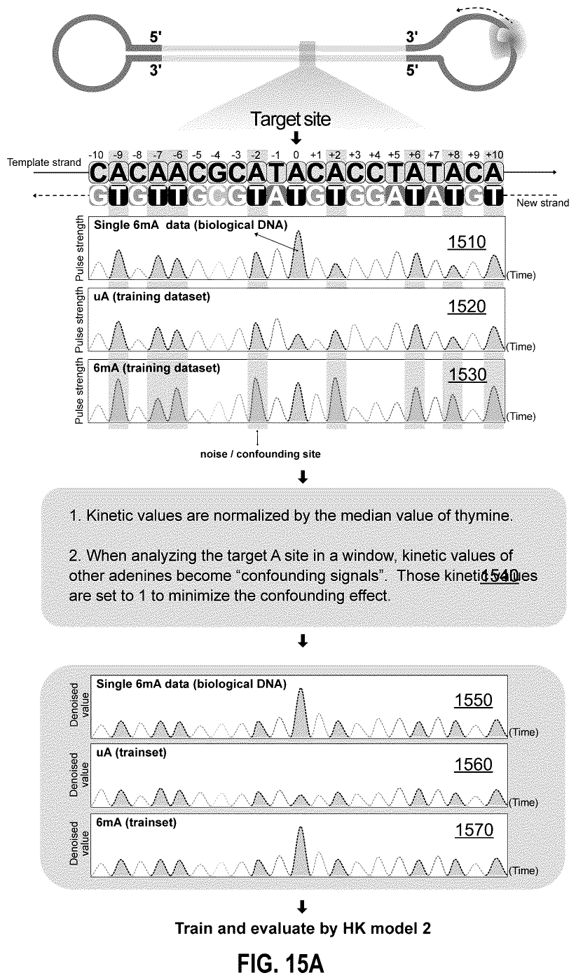

FIG. 13A shows 6 mA methylation levels determined by HK model 2.The x-axis shows the type of microbial DNA. The y-axis shows the predicted 6 mA methylation level at GATC sites. The predicted median 6 mA methylation levels related to GATC motifs were 95% in both E. coli and S. enterica, whereas 2%, 1%, 2%, and 2%, for B. subtills, E. faecalis, L. mono, and S. aureus, respectively. The results were excellently matched with the expectation.

FIG. 13B shows de novo motif analysis related to 6 mA modifications. The x-axis shows the relative distance to the observed 6 mA site. The y-axis shows bits (a measure of entropy). Interestingly, apart from the well-known GATC motif, their respective characteristic motifs associated with 6 mA were determined to be ACA(N)₈TG, AAGA(N)₅CTC, CCAA(N)₇TTG, GCA(N)₇TGC, TA(N)₆TA, CAGAG for B. subtills, E. coli, E. facecalis, L. mono, S. aureus, and S. enterica, respectively, which were also comparable with the previous studies^{17,18}. These results further suggested that the 6 mA detector could be a useful tool for 6 mA analysis in real biological samples.

We further evaluated the performance in two situations of signal patterns: (1) a region with a few modifications, named as sparse signal pattern; (2) a region with many modifications, referred to as dense signal pattern. In one embodiment, a single measurement window (e.g., a 21-nt window size) may contain multiple modified sites, potentially causing a confounding effect from neighboring modifications and resulting in a model that cannot be generalized well.

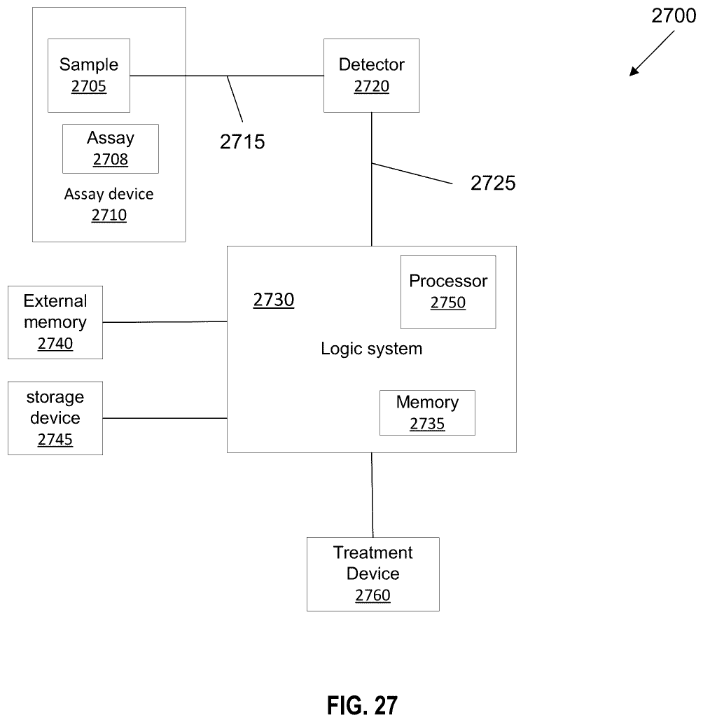

FIG. 27 illustrates sparse and dense signal patterns with a 6 mA modification. Sparse signal pattern can be defined as a region containing, but not limited to, no more than 2, 3, 4, or 5 modifications per 100 nucleotides. Dense signal pattern can be defined as a region containing, but not limited to, more than 5, 6, 7, or 8 modifications per 100 nucleotides. In some embodiments, multiple base modification may include different types of base modifications, such as 4 mC, oxoG, 5 mC, or any other modification described herein.

One example of sparse signal pattern may be generated using M.SssI methyltransferase that only methylates cytosines at CpG context. The frequency of CpG in a human genome is approximately 1 out of 100 nucleotides. One example of dense signal pattern may be generated using the whole genome amplification with the presence of methylated adenines. All adenine sites in amplified DNA produce would be methylated and the frequency of adenine in a human genome is approximately 30 out of 100 nucleotides.

To make it possible to detect either single or multiple 6 mA sites present in a measurement window using one generic model, we developed a new normalization strategy to make the pattern of multiple 6 mA signals comparable with that of single 6 mA signals.

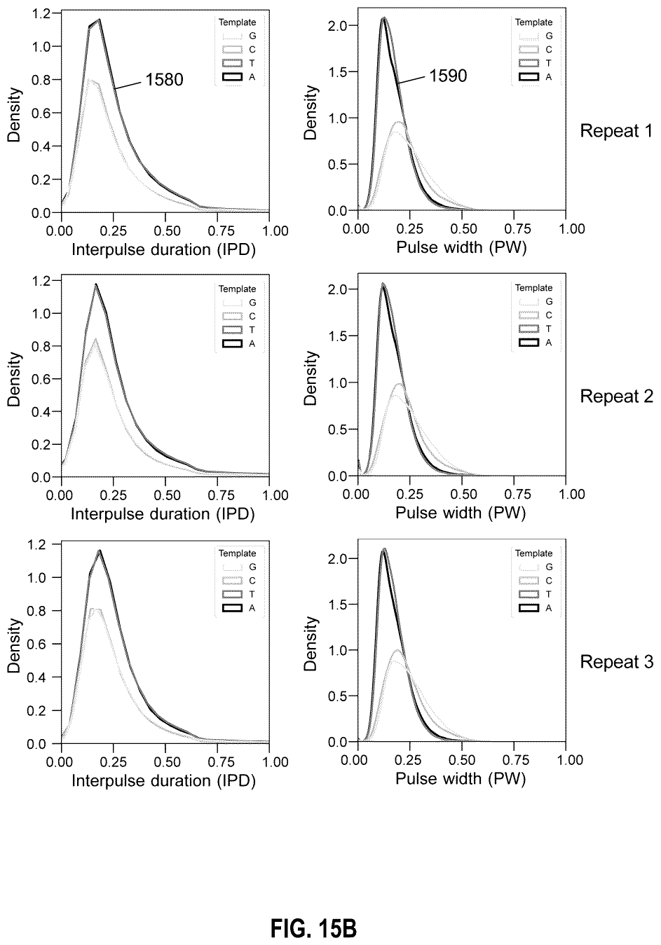

FIG. 15A illustrates the distribution of kinetics features in a measurement window between before and after signal normalization in different datasets. Graph 1510 shows single 6 mA data in biological samples (sparse signal pattern, with one 6 mA site in a measurement window). Graph 1520 shows uA in training dataset (without 6 mA site in a measurement window). Graph 1530 shows 6 mA in training (dense signal pattern, with multiple 6 mA sites in a measurement window). Graph 1530 and graph 1510 have distinctly different signal patterns. Hence, training on a dense signal pattern may result in erroneous classifications when analyzing a biological sample. To solve this, we developed a method for correcting the kinetic signal in a measurement window. We applied a denoising processing 1540 for this purpose (referred to as denoiser) but using a median value of thymine signals.

FIG. 15B shows the density distributions of kinetic features in different bases of templated DNA on the basis of PacBio Sequel II kit 2.0. Three randomly sampled data from one WGA-uA dataset were analyzed (Repeat 1, 2, and 3). We observed that the distributions of kinetic values were similar between unmodified adenine and thymine (e.g., peaks 1580 and 1590).

For the denoising processing 1540, all kinetic values in a measurement window were divided by the median kinetic value of thymines. Therefore, the normalized kinetic signals of uA exhibited a distribution with a mean of 1. As the neighboring 6 mA sites may confound the target site analysis, the kinetic values regarding those “confounding sites” were set to 1, resembling with uA distribution to minimize the confounding effect during the training.

Graph 1550 shows the denoised single 6 mA biological data. Graph 1560 shows the denoised uA training data. Graph 1570 shows the denoised 6 mA training data. Graph 1570 resembles graph 1550 after denoising.

FIGS. 11B to 11E used the HK model 2 trained by the normalized data from WGA-6 mA and WGA-uA datasets to establish the 6 mA detector. As a result, the 6 mA detector could reach an AUC of 0.99. FIGS. 12A and 12B used the HK model 2 trained by the normalized data from WGA-4 mC and WGA-uC datasets to establish the 4 mC detector. The 4 mC detector was superior to the conventional analysis (AUC: 0.98 versus 0.92).

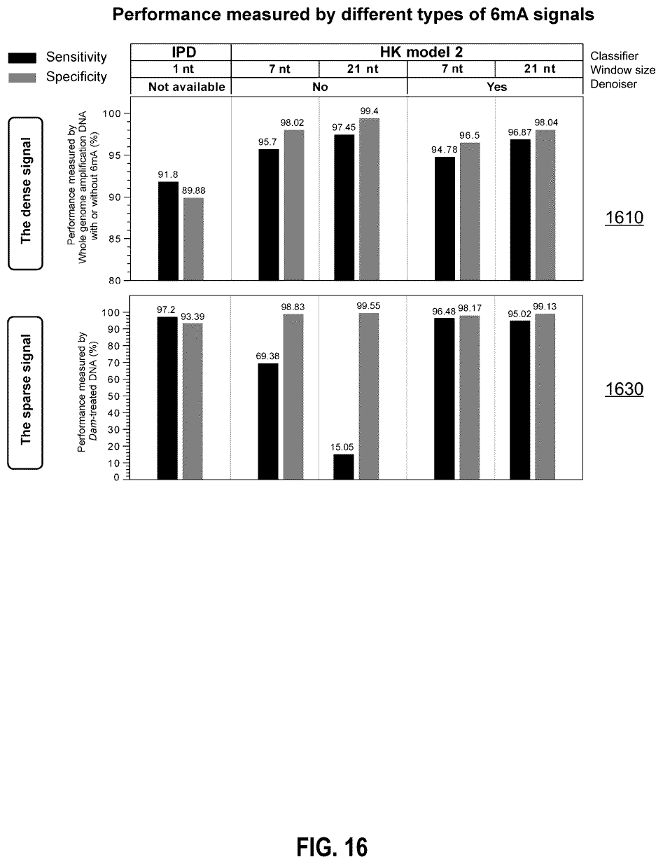

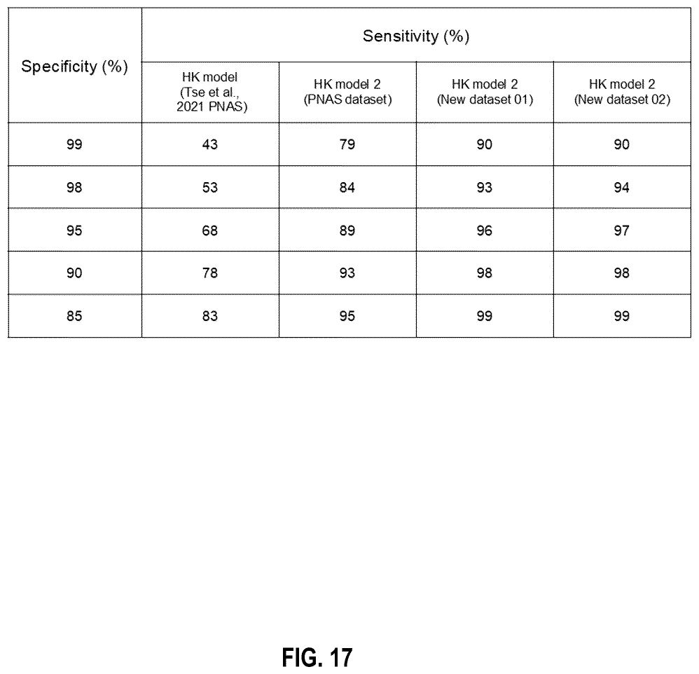

FIG. 16 shows the performance of classifiers of 6 mA using different types of 6 mA signals. The columns show the different types of classifiers, including using IPD and HK model 2. For HK model 2, measurement size windows of 7 nt and 21 nt were used. For each measurement window size, models trained with the denoiser and no denoiser were both used. The rows show the results for classifying different signals. For each pair of columns, the first column shows sensitivity, and the second column shows specificity. Row 1610 shows results from a dense signal. Row 1630 shows results for a sparse signal, prepared by Dam methyltransferase-treated DNA. The sparse signal patterns prepared by Dam treatments introduced an 6 mA to the GATC motifs.

As shown row 1610, without using the denoiser, the sensitivity and specificity of HK model 2 (6 mA detector) in the dense signal data increased when enlarging measurement window size. Compared with IPD-based method (sensitivity: 91.8%; specificity: 89.88%), HK model 2 resulted in higher sensitivity and specificity of 95.7% and 98.8%, 97.45% and 99.4% in 7-nt and 21-nt window size, respectively, without using the denoiser.

However, for the sparse signal patterns prepared by Dam treatment, the decrease in sensitivities was to 69.38% and 15.05% in 7-nt and 21-nt window sizes, respectively. These results suggested reducing window size could in part alleviate the overfitting issue.

After incorporating the denoiser into the HK model 2, a robust performance in terms of the sensitivity and specificity across all test datasets had been achieved. For example, in row 1610, the sensitivity and specificity were 96.87% and 98.04%, respectively, in the data with a 21-nt window with denoiser for dense signal patterns obtained from whole genome amplification. In row 1630, the sensitivity and specificity were 95.02% and 99.13%, respectively, in the data with a 21-nt window with denoiser for sparse signal pattern obtained from Dam treatment. These test results suggested that the denoiser developed in this disclosure can be used for 6 mA detection with high adaptability and stability.

### 4. Detecting Other Methylations

With an increased throughput of Lig-5 hmCG, one could directly train a multiclass model for differentiating uC, 5 mC, and 5hmC, by preparing the various training datasets mediated by DNA ligation (FIG. 8B). Because 5 fC and 5 caC were reported to be 10 to 10,000-fold less abundant than 5 hmC in genomic DNA across various tissues and cells examined²¹, the actual impact of residual 5 fC and 5 caC on the analysis of real biological samples may be small. The low amount of methylation types other than 5 mC and 5 hmC in genomic DNA is at least partly be supported by the consistent results observed between the 5 xC and 5 hmC patterns in brain tissues measured by HK model 2, BS-seq, and TAB-seq.

### F. Applications of Model

We have demonstrated that HK model 2 exhibited better performance with versatile functions in determining various types of base modifications.

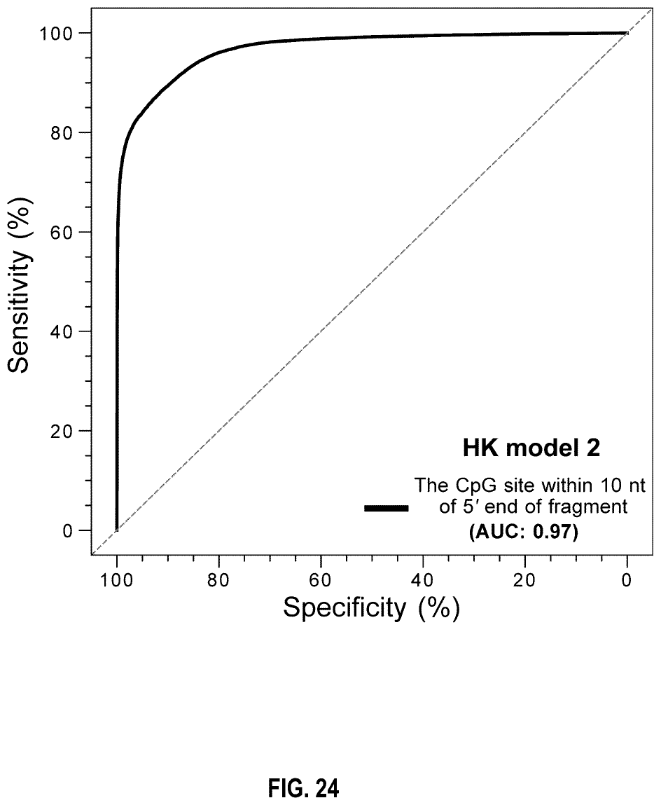

FIG. 24 shows the different sensitivities at given specificities for different double-stranded models. HK model 2 outperforms HK model 1 at all specificities for all datasets.

FIG. 18 shows different sensitivities at given specificities for different single-stranded models. HK model 2 can be used to detect different methylation types.

We next set out to investigate what is the potential impact on the clinical and biological applications. Choy et al. recently demonstrated that on the basis of HK model 1, the analysis of methylation patterns of cfDNA molecules in patients with hepatocellular carcinoma (HCC) enabled the detection of HCC³. Choy et al. introduced the HCC methylation score that was derived from comparing the methylation pattern of each long cfDNA molecule deduced by HK model 1 with the methylation patterns of reference tissues (e.g., HCC tumor tissues and normal tissues)³. Using HK model 2, we reanalyzed Choy et al.'s dataset including cfDNA molecules with 1 to 6 CpG sites and calculated the HCC methylation score.

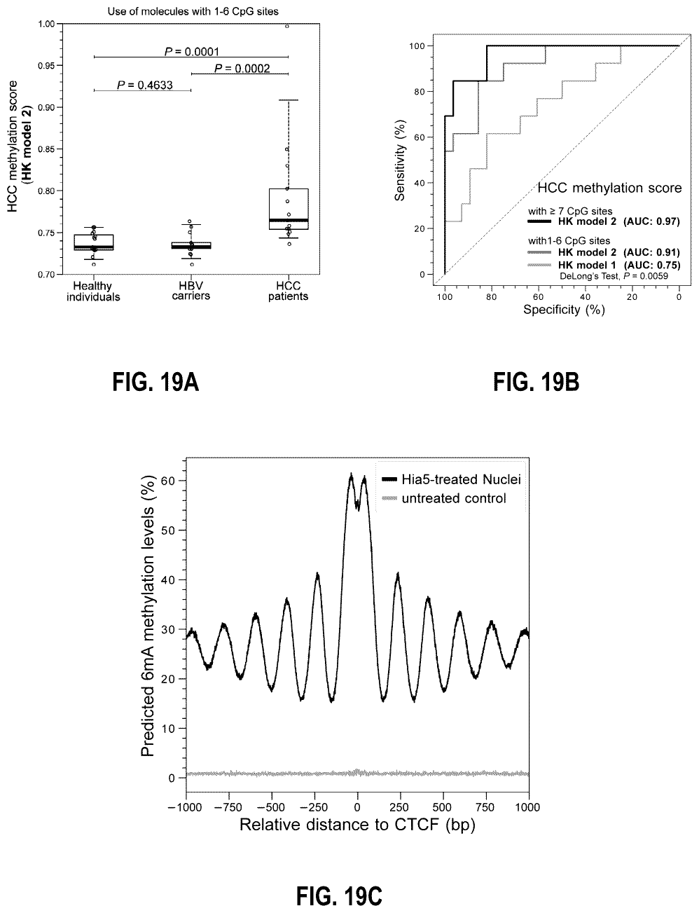

FIG. 19A shows HCC methylation scores determined by HK model 2 in healthy individuals, HBV carriers, and HCC patients using sequenced DNA molecules with 1 to 6 CpG sites. The x-axis shows the type of individual. The x-axis shows the HCC methylation score determined by HK model 2. We observed that HCC methylation scores in HCC patients (median: 0.764; IQR: 0.751-0.802) were significantly higher than those in non-HCC individuals (i.e., healthy individuals and HBV carriers) (median: 0.733; IQR: 0.729-0.745) (P value=0.0001; Mann-Whitney U test). FIG. 19A shows HCC patients have methylation scores that are statistically significantly different from non-HCC individuals.

The HCC methylation score is described in US 2023/0279498 A1, filed Nov. 23, 2022, which is incorporated by reference in its entirety for all purposes. Briefly, to assess the risk of having HCC for an individual, we adapted tissue-of-origin analysis by comparing the methylation patterns of plasma DNA molecules with the methylation profile of HCC tumor tissue. Tissue methylomes of HCC tumor tissue was obtained from a previous study.

Firstly, a score, S(HCC), which reflected the similarity between a DNA molecule and the HCC tumor tissue, was calculated by the following formula:

$$S ( H C C ) = \frac{{Σ}_{j = 1}^{n} ( 1 - \vert {P}_{j} - {r}_{j , HCC} \vert )}{n} ,$$

where Pⱼ is the methylation status for a CpG site j in a plasma DNA molecule, r_{j,HCC} is the methylation index for the corresponding CpG site in the reference methylome of tumor tissue, and n is the total number of CpG sites in a plasma DNA molecule.

Similarly, another score, S(BC), was calculated to determine the similarity of methylation pattern between a DNA molecule and the buffy coat by:

$$S ( B C ) = \frac{{Σ}_{j = 1}^{n} ( 1 - \vert {P}_{j} - {r}_{j , BC} \vert )}{n} .$$

Finally, both S(HCC) and S(BC) were integrated into a metric called HCC methylation score by the following formula:

$$HCC methylation score = \frac{{Σ}_{k = 1}^{T} [ 1 + ( {S}_{H C C} - {S}_{B C} ) ]}{T} ,$$

where T is the total number of plasma DNA molecules being analyzed in one individual. The higher the HCC methylation score, the more likely a testing sample would have HCC.

FIG. 19B shows ROC curves of using HCC methylation score for classifying individuals with and without HCC on the basis of molecules with 1 to 6 CpG sites or at least 7 CpG sites. The x-axis is specificity. The y-axis is sensitivity. The HCC methylation score based on HK model 2 leads to a higher AUC in distinguishing between individuals with and without HCC (0.91), compared with that based on HK model 1 (AUC: 0.75). The performance of HCC detection could be further improved to 0.97 if we used the dataset including cfDNA molecules with at least 7 CpG sites. These results suggested that we can not only reproduce the previous findings using HK model 2, but also achieve better performance.

Another possible application of our 6 mA detector is to infer the nucleosome positioning. The 6 mA modifications may be differentially introduced into the chromatin depending on its accessibility states via DNA adenine methyltransferases (e.g., Hia5)¹². The HK model 2 based 6 mA detector was used to analyze the SMRT-seq result of the human nuclei (K562 cell line) which was treated by Hia5¹². We then identified the nucleosome positioning in genomic regions near CTCF binding sites (i.e., CCCTC-binding factor), which is known to be flanked with well-organized nucleosomal patterns¹². We analyzed the 6 mA signals within 1 kb upstream and downstream relative to CTCF binding sites.

FIG. 19C shows patterns of 6 mA levels in genomic sites relative to CTCF binding sites. The x-axis shows relative distance to CTCF binding sites. The y-axis shows predicted 6 mA methylation levels.

As seen in FIG. 19C, the 6 mA levels in genomic sites relative to CTCF binding sites displayed periodic signals with an interval of approximately 180 bp, resembling nucleosomal arrays. We envisioned that the distance between two consecutive peaks of 6 mA levels could facilitate the determination of nucleosome positioning, and the magnitude of 6 mA levels might indicate the openness of chromatin states. For example, a higher methylation level may indicate a higher openness or a lower occupation of protein. Nucleosome position may be determined by measuring the distance between two consecutive peaks. Such applications with methylation level and binding sites are discussed in Stergachis et al., Science, Vol. 368, Issue 6498 (2020), which is incorporated by reference in its entirety for all purposes.

### G. Discussion

We have developed an enhanced deep learning framework, named HK model 2, for analyzing multiple base modifications of DNA molecules sequenced by SMRT-seq. The sensitivities of HK model 2 for 5 mC, 5 hmC, and 6 mA detection could be up to 98%, 90%, and 99%, respectively, at an overall specificity of over 90%. Such a framework has been implemented using a hybrid architecture of deep learning models including CNN and transformers. CNN can effectively capture the local feature patterns in a measurement window through the convolutional process, and transformers can learn global feature patterns through the ‘self-attention’ mechanism²⁰. Furthermore, preparing the deep learning model may include training dataset preparation and data processing of the input features (e.g., signal normalization).

In this study, we have devised solutions regarding training dataset preparation and signal processing, depending on the target type of base modification. For example, for 5 mC detection, the DNA end repair process was rearranged prior to the M.SsssI treatment, reducing the contamination of unmodified cytosines present in the training dataset of methylated DNA. Such a design of experimental protocol has improved model performance, typically for those CpG sites proximal to the 3′ ends of DNA fragments. For 5 hmC detection, we developed an novel approach based on DNA ligation to obtain a training dataset with a high purity of 5 hmC modification. Moreover, we extended the capability of HK model 2 to 6 mA detection, using a unique signal normalization to minimize the potential confounding effect of neighboring 6 mA sites. Therefore, HK model 2 can have equally good performance in detecting the 6 mA modification, regardless of whether a single or multiple 6 mA modifications are present in a measurement window.

In addition to the performance evaluation of HK model 2, we explored the potential impact on the applications of using the generic and improved model framework. For instance, the long cfDNA has more CpG sites, harboring the enriched tissue-specific molecular information^{2,3}, but often having relatively low subread depths. Because of the enhanced accuracy of 5 mC detection for sequenced molecules with low subread depths, the tissue-of-origin analysis of recently identified long cfDNA molecules using HK model 2 should be superior to using HK model 1. Indeed, the performance of HCC detection has been greatly enhanced up to an AUC of 0.97 with HK model 2. For 6 mA detection, we accurately determined the 6 mA modification in real biological samples (i.e., microbial DNA), which usually sparsely contain a single 6 mA site in a measurement. On the other hand, we could determine the jagged ends of cfDNA molecules and the accessibility profile of native chromatin fibers in nuclei in which the 6 mA modifications were artificially introduced, usually occurring across many nearby positions. These data further demonstrated the robustness of the structure of the HK model 2 developed in this study.

Taken together, the HK model 2 is a versatile and improved approach for detecting multiple base modifications using single molecule real-time sequencing, augmenting current efforts in developing non-invasive cancer detection, as well as dissecting chromatin structures.

### H. Example Method of Model Training

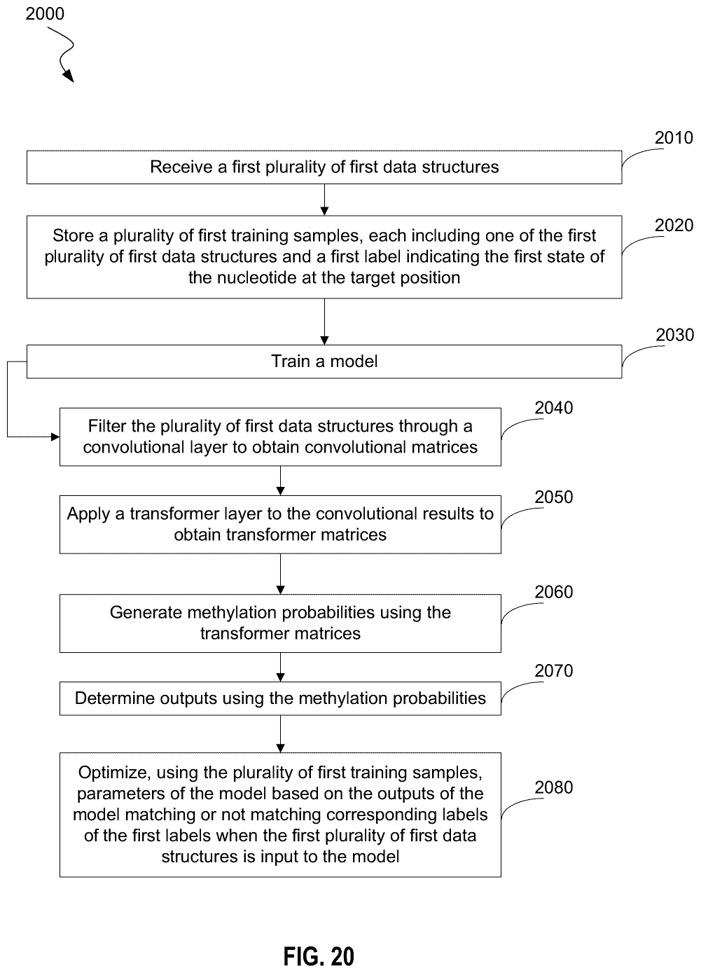

FIG. 20 is a flowchart of an example process 2000 for detecting a methylation of a nucleotide in a nucleic acid molecule. Process 2000 may be for training a model to detect a methylation. In some implementations, one or more process blocks of FIG. 20 may be performed by any system described herein, including system 2700.The methylation may be any methylation described herein, including 5 mC (5-methylcytosine) or 6 mA (N6-methyladenine).

At block 2010, a first plurality of first data structures is received. Each first data structure of the first plurality of data structures may correspond to a respective window of nucleotides sequenced in a respective nucleic acid molecule of a plurality of first nucleic acid molecules. Each of the first nucleic acid molecules may be sequenced by measuring pulses in a signal corresponding to the nucleotides. The methylation may have a known first state in a nucleotide at a target position in each window of each first nucleic acid molecule. The known first state may be whether the methylation is present or absent. Each first data structure may include values for one or more signal properties at positions within the respective window. FIG. 2A and FIG. 2B show examples of first data structures. The plurality of known first states may comprise known 5 mC, 5 hmC, 6 mA, 4 mC, 5 fC, 5 caC, 1 mA, 3 mA, 7 mA, 3 mC, 2 mG, 6 mG, 7 mG, 3 mT, and 4 mT states.

The plurality of first nucleic acid molecules may include single-stranded nucleic acid molecules. The plurality of first nucleic acid molecules may be obtained by methylating sites after repairing damage of sonicated nucleic acid molecules or after end repairing the sonicated nucleic acid molecules (e.g., as described with FIG. 18).

The signal may be an optical signal (e.g., fluorescence, chemiluminescence, or photometric signal) or an electrical signal. The optical signal may be from single molecule, real-time sequencing. The signal may result from the nucleotides or tags associated with the nucleotides.

The electrical signal may be from nanopore sequencing. The electrical signal may be a current, voltage, resistance, inductance, capacitance, or impedance. Electrical signals are described in US Patent Publication No. 2022/0328135 A1, filed Apr. 12, 2022, the entire contents of which are incorporated herein by reference for all purposes.

Each window of each first data structure may include 4 or more consecutive nucleotides, including 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 200, 500, or within any range between and including any two of the numbers or more consecutive nucleotides. Each window may have the same number of consecutive nucleotides. For example, each window may include 21 consecutive nucleotides upstream of the nucleotide at the target position and 21 consecutive nucleotides downstream of the nucleotide at the target position. Each window may have a different number of consecutive nucleotides upstream of the nucleotide at the target position than the number of consecutive nucleotides downstream of the nucleotide at the target position.

The target position may be the center of the respective window. For a window spanning an even number of nucleotides, the target position may be the position immediately upstream or immediately downstream of the center of the window. In some embodiments, the target position may be at any other position of the respective window, including the first position or the last position. For example, if the window spans n nucleotides of one strand, from the 1st position to the nth position (either upstream or downstream), the target position may be at any from the 1st position to the nth position.

The windows may be overlapping. Each window may include nucleotides on a first strand of the first nucleic acid molecule and nucleotides on a second strand of the first nucleic acid molecule. The first data structure may also include for each nucleotide within the window a value of a strand property. The strand property may indicate the nucleotide being present or either the first strand or the second strand. The window may include nucleotides in the second strand that are not complementary to a nucleotide at a corresponding position in the first strand. In some embodiments, all nucleotides on the second strand are complementary to the nucleotides on the first strand. In some embodiments, each window may include nucleotides on only one strand of the first nucleic acid molecule.

The one or more signal properties may include the sequence context. The one or more signal properties may include an identity of the nucleotide (e.g., A, T, C, or G) for each nucleotide within each window. The one or more signal properties may also include, for each nucleotide within each window, a position of the nucleotide within the sample nucleic acid molecule, a width of a pulse corresponding to the nucleotide, and/or an interpulse duration (IPD) representing a time between the pulse corresponding to the nucleotide and a pulse corresponding to a neighboring nucleotide.

Each data structure of the plurality of first data structures may exclude first nucleic acid molecules with an IPD or width below a cutoff value. For example, only first nucleic acid molecules with an IPD value greater than a 10^{th} percentile (or a 1^{st}, 5^{th}, 15^{th}, 20^{th}, 30^{th}, 40^{th}, 50th, 60^{th}, 70^{th}, 80^{th}, 90^{th}, or 95^{th} percentile) may be used. The percentile may be based on data from all nucleic acid molecules in a reference sample or reference samples. The cutoff value of the width may also correspond to a percentile.

The width of the pulse may be the width of the pulse at half the maximum value of the pulse. The interpulse duration may be the time between the maximum value of the pulse associated with the nucleotide and the maximum value of the pulse associated with the neighboring nucleotide. The neighboring nucleotide may be the adjacent nucleotide. The properties may also include a height of the pulse corresponding to each nucleotide within the window. The properties may further include a value of a strand property, which indicates whether the nucleotide is present on the first strand or the second strand of the first nucleic acid molecule.

The position may be a nucleotide distance relative to the target position. The position may be +1 when the nucleotide is one nucleotide away from the target position in one direction, and the position may be −1 when the nucleotide is one nucleotide away from the target position in the opposite direction.

The signal properties may include a vector including a first segment statistical value of a segment of the electrical signal corresponding to the nucleotide. Properties may include a first region statistical value of the electrical signal in a region of the nucleic acid molecule equal to or larger than the window.

The first segment statistical value may represent a mean of the segment of the electrical signal corresponding to the nucleotide. In some embodiments, the first segment statistical value may represent a variation (e.g., standard deviation) of the electrical signal of the segment of the electrical signal corresponding to the nucleotide. In embodiments, the first segment statistical value may represent a normalized value of a mean of the segment of the electrical signal corresponding to the nucleotide. Normalization may include rescaling so that the first segment statistical value is in a certain range (e.g., a range from 0 to 1). Normalization may include using the median value, the mean value, and/or deviations for part or all of the nucleotide strand.

The vector may include a second segment statistical value representing a variation of the segment of the electrical signal corresponding to the nucleotide. The vector may include a third segment statistical value representing a normalized value of the first segment statistical value.

The first region statistical value may represent a mean or median of the electrical signal in the region. In embodiments, the first region statistical value may represent a median or mean of an absolute value of a variation of the electrical signal from the mean or median of the electrical signal in the region. The variation may be a standard deviation. In some embodiments, the first region statistical value may be optional.

The input data structure may further include a second region statistical value representing a median or mean of an absolute value of a variation of the electrical signal from the mean or median of the electrical signal in the region.

The first plurality of first data structures may include 5,000 to 10,000, 10,000 to 50,000, 50,000 to 100,000, 100,000 to 200,000, 200,000 to 500,000, 500,000 to 1,000,000, or 1,000,000 or more first data structures. The plurality of first nucleic acid molecules may include at least 1,000, 10,000, 50,000, 100,000, 500,000, 1,000,000, 5,000,000, or more nucleic acid molecules. As a further example, at least 10,000 or 50,000 or 100,000 or 500,000 or 1,000,000 or 5,000,000 sequence reads can be generated.

The plurality of first nucleic acid molecules may include molecules with adaptors, as described in the disclosure. A subset of the plurality of first nucleic acid molecules may include extended nucleic acid molecules. The extended nucleic acid molecule may include a sample nucleic acid molecule and an adaptor. The adaptor may have a known sequence. The respective window of nucleotides may include at least one nucleotide in the adaptor. Details of using adaptors are discussed elsewhere in this disclosure.

The first data structures may include sparse signal patterns (e.g., less than 1 methylation for 25 nucleotides). The methylation may be 6 mA, 4 mC, or any methylation described herein. The window may include 21 or fewer consecutive nucleotides. Each first data structure may include values for one or more signal properties corresponding to a methylated nucleotide for no more than one nucleotide in the respective window. Each first nucleic acid molecule may include no more than one methylated nucleotide in any window corresponding to any first data structure in the first plurality of first data structures. For example, the plurality of first nucleic acid molecules may include first nucleic acid molecules having ends repaired with a methylated nucleotide (e.g., as described with FIG. 30). In some embodiments, the plurality of first nucleic acid molecules may include first nucleic acid molecules treated with DNA adenine methyltransferase (Dam) enzyme. In some embodiments, enzymes used for treatment may include exocyclic amino methyltransferases and endocyclic methyltransferases. Exocyclic amino methyltransferases may include Dam and CcrM. Endocyclic methyltransferases may include Dcm.

In some embodiments, the values for the one or more signal properties for a portion of the plurality of first data structures may include one or more values corresponding to one or more nucleotides that are determined using signal properties measured for nucleotides other than the one or more nucleotides. For example, the one or more nucleotides may be adenines, and the nucleotides other than the one or more nucleotides are thymines. A statistical measure (e.g., mean, median, or mode) of one nucleotide type may be used to normalize other nucleotide types (as described with the denoiser of FIG. 15A). Reducing the number of methylated nucleotides in a window may involve using the statistical measure of a signal property in place of what was measured for the methylated nucleotide.

At block 2020, a plurality of first training samples is stored. Each first training sample may include one of the first plurality of first data structures and a first label indicating the first state of the nucleotide at the target position. Storage may be on any computer readable medium described herein.

At block 2030, a model is trained. The model may be trained by blocks 2040-2080.

At block 2040, the plurality of first data structures is filtered through a convolutional layer to obtain convolutional matrices. Block 2040 may correspond to stage 108 in FIG. 1. The respective convolutional matrices may have lower dimensionality than the respective first data structure.

The convolutional layer may be part of a convolutional neural network (CNN). The CNN may include a set of convolutional filters configured to filter the first plurality of first data structures. The filter may be any filter described herein. The number of filters for each layer may be from 10 to 20, 20 to 30, 30 to 40, 40 to 50, 50 to 60, 60 to 70, 70 to 80, 80 to 90, 90 to 100, 100 to 150, 150 to 200, or more. The kernel size for the filters can be 2, 3, 4, 5, 6, 7, 8, 9, 10, 11, 12, 13, 14, 15, from 15 to 20, from 20 to 30, from 30 to 40, or more. The CNN may include an input layer configured to receive the filtered first plurality of filter data structures. The CNN may also include a plurality of hidden layers including a plurality of nodes. The first layer of the plurality of hidden layers may be coupled to the input layer.

In some embodiments, the model may include a recurrent neural network (RNN). The RNN may be in place of the CNN, and the results from the RNN may be used instead of the convolutional matrices.

At block 2050, a transformer layer is applied to the convolutional matrices to obtain

transformer matrices. Block 2050 may correspond to stage 112 in FIG. 1, and the transformer layer may be any transformer layer described herein. Applying the transformer layer may include generating a plurality of attention scores that quantify a relevance among positions of the convolutional matrices.

In some embodiments, block 2040 is optional and the plurality of first data structures may be fed directly to the transformer layer.

The transformer layer may include a number of parameters for training. The parameters may include the number of multiple-head self-attention, QKV biases (queries, keys, and values in attention mechanisms), and a ratio of multilayer perceptron.

Generating the plurality of attention scores may include using a plurality of multiple-head self-attentions. The number of multiple-head self-attentions may be 1, 2, 3, 4, 5, 6, 7, 8, 9, 10, 15, 20, 30, 40, or 50, or within any range between and including any two of the numbers.

K, Q, and V may be vectors filled with weights. The inputs may be multiplied with a set of weights for K, Q, and V. The multiplications may be conducted many times, such as, but not limited to, 1, 2, 3, 4, 5, 6, 7, 8, 9, 10, 15, 20, 25, 30, 35, 40, 45, 50, 100, 200, 300, 400, or 500 times, or within any range between and including any two of the numbers.

Applying the transformer layer may include normalizing the convolutional matrices. A softmax function may be used to determine attention scores.

At block 2060, methylation probabilities are generated using the transformer matrices. Generating the methylation probabilities may include applying one or more neural network layers to the transformer matrices. Applying the one or more neural network layers may include performing multiplication by weights or additions by biases.

At block 2070, outputs are determined using the methylation probabilities. The outputs

may be whether the methylation is present. In some embodiments, the outputs may be “0” or “1” or other binary classification based on a comparison of the probabilities to cutoff values. For example, a methylation probability greater than the cutoff value may result in an output of “1”.

At block 2080, parameters of the model are optimized, using the plurality of first training samples, based on the outputs of the model matching or not matching corresponding labels of the first labels when the first plurality of first data structures is input to the model. An output of the model specifies whether the nucleotide at the target position in the respective window has the methylation. The parameters of the model may include the plurality of attention scores.

As part of training a machine learning model, the parameters of the machine learning model (such as weights, thresholds, e.g., as may be used for activation functions in neural networks, etc.) can be optimized based on the training samples (training set) to provide an optimized accuracy in classifying the methylation of the nucleotide at the target position. Various form of optimization may be performed, e.g., backpropagation, empirical risk minimization, and structural risk minimization. A validation set of samples (data structure and label) can be used to validate the accuracy of the model. Cross-validation may be performed using various portions of the training set for training and validation. The model can comprise a plurality of submodels, thereby providing an ensemble model. The submodels may be weaker models that once combined provide a more accurate final model.

Process 2000 may include additional implementations, such as any single implementation or any combination of implementations described herein and/or in connection with one or more other processes described elsewhere herein.

Although FIG. 20 shows example blocks of process 2000, in some implementations, process 2000 may include additional blocks, fewer blocks, different blocks, or differently arranged blocks than those depicted in FIG. 20. Additionally, or alternatively, two or more of the blocks of process 2000 may be performed in parallel.

### I. Example Method of Detecting Methylation

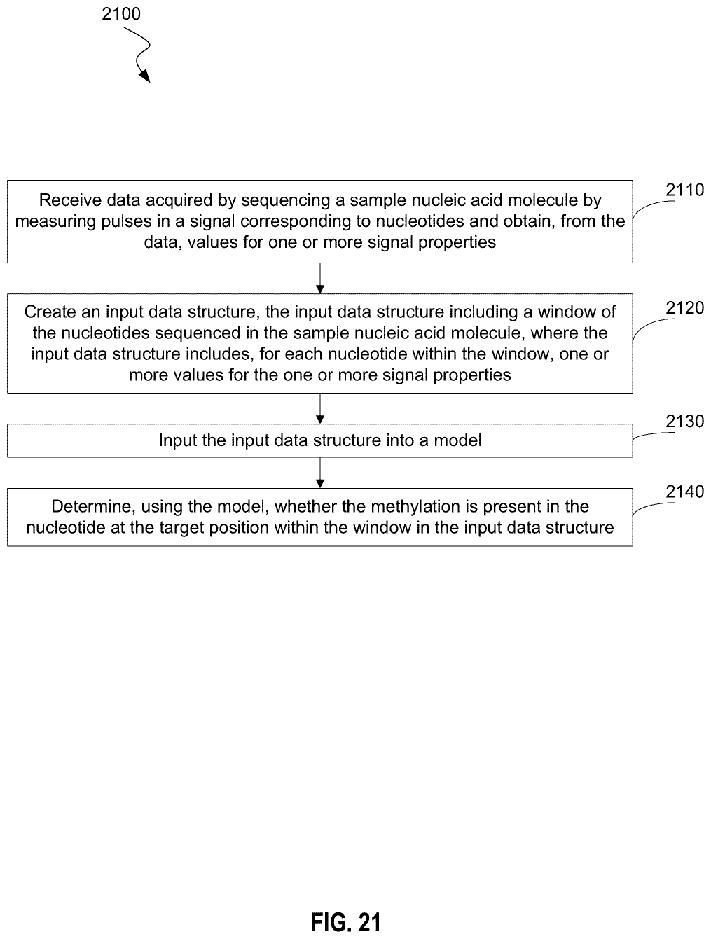

FIG. 21 is a flowchart of an example process 2100 for detecting a methylation of a

nucleotide in a nucleic acid molecule. In some implementations, one or more process blocks of FIG. 21 may be performed by system 2700 or any system described herein. The methylation may be any methylation described herein, including 5 mC (5-methylcytosine), 6 mA (N6-methyladenine), 5 hmC, 4 mC, 5 fC, 5 caC, 1 mA, 3 mA, 7 mA, 3 mC, 2 mG, 6 mG, 7 mG, 3 mT, or 4 mT.

At block 2110, data acquired by sequencing a sample nucleic acid molecule by measuring pulses in a signal corresponding to nucleotides are received. The signal may be an optical signal or an electrical signal. The signal may be any signal described herein, including with process 2000. Values for one or more signal properties are obtained from the data. Process 2100 may include sequencing the sample nucleic acid molecule by any sequencing technique described herein. The sample nucleic acid molecule may be single stranded or double stranded.

In some embodiments, data may be acquired by sequencing an extended nucleic acid molecule. The extended nucleic acid molecule may include the sample nucleic acid molecule and an adaptor. The adaptor may have a known sequence and may be any adaptor described herein.

The one or more signal properties may include an identity of the nucleotide for each nucleotide within each window. The one or more signal properties may also include, for each nucleotide within each window, a position of the nucleotide within the sample nucleic acid molecule, a width of a pulse corresponding to the nucleotide, and/or an interpulse duration representing a time between the pulse corresponding to the nucleotide and a pulse corresponding to a neighboring nucleotide. The one or more signal properties may include the sequence context. The signal properties may include any signal properties described herein. In some embodiments, the window of nucleotides may include at least one nucleotide in an adaptor. The signal properties may be the signal properties described with process 2000 or any signal properties described herein.

At block 2120, an input data structure is created. The input data structure may include a window of the nucleotides sequenced in the sample nucleic acid molecule. The input data structure may include, for each nucleotide within the window, one or more values for the one or more signal properties. The window of the input data structure may have similar properties as the window of each first data structure in process 2000.

The nucleotides within the window may or may not be aligned to a reference genome. The nucleotides within the window may be determined using a circular consensus sequence (CCS) without alignment of the sequenced nucleotides to a reference genome. The nucleotides in each window may be identified by the CCS rather than aligning to a reference genome. In some embodiments, the window may be determined without a CCS and without alignment of the sequenced nucleotides to a reference genome.

The nucleotides within the window may be enriched or filtered. The enrichment may be by an approach involving Cas9.The Cas9 approach may include cutting a double-stranded DNA molecule using a Cas9 complex to form a cut double-stranded DNA molecule and ligating a hairpin adaptor onto an end of the cut double-stranded DNA molecule. The filtering may be by selecting double-stranded DNA molecules having a size within a size range. The nucleotides may be from these double-stranded DNA molecules. Other methods that preserve the methylation status of the molecules may be used (e.g., methyl-binding proteins).

At block 2130, the input data structure is inputted into a model. The model may be trained by process 2000 or any method described herein. The model may include the framework described with FIG. 1 in section I.A. The model may include one or more transformer layers, including any transformer layers described herein. In some embodiments, the transformer layer may generate a plurality of attention scores that quantify a relevance among positions of data in the input data structure. In some embodiments, the transformer layer may generate a plurality of attention scores that quantify a relevance among positions of data among convolutional matrices from filtering the input data structure through a convolutional layer.

At block 2140, whether the methylation is present in the nucleotide at the target position within the window in the input data structure is determined using the model.

In some embodiments, process 2100 may further include determining the methylation type is a first type among a plurality of types (e.g., as described with FIGS. 8A, 8B, and 8C). Determining whether the methylation is present may include determining the methylation is present and determining the methylation is the first type. For example, each type of the plurality of types may be one of 5 mC, 5 hmC, 6 mA, or any methylation described herein. In some embodiments, the determination of the methylation type may occur at the same time as the determination of whether the methylation is present. For example, a training set for the methylation type may be sufficient for a desired accuracy. The training set may include 1 to 5 million, 5 to 10 million, 10 to 15 million, 15 to 20 million, or over 20 million methylated sites of a particular type.

The input data structure may be one input data structure of a plurality of input data structures. Each input data structure may correspond to a respective window of nucleotides sequenced in a respective sample nucleic acid molecule of the plurality of sample nucleic acid molecules. The plurality of sample nucleic acid molecules may be obtained from a biological sample of a subject. The biological sample may be any biological sample described herein. Process 2100 may be repeated for each input data structure. The method may include creating the plurality of input data structures. The plurality of input data structures may be inputted into the model. Whether a methylation is present in a nucleotide at the target location in the respective window of each input data structure may be determined using the model. The plurality of sample nucleic acid molecules may be single stranded, double stranded, or a combination.

Each sample nucleic acid molecule of the plurality of sample nucleic acid molecules may have a size greater than a cutoff size. For example, the cutoff size may be 100 bp, 200 bp, 300 bp, 400 bp, 500 bp, 600 bp, 700 bp, 800 bp, 900 bp, 1 kb, 2 kb, 3 kb, 4 kb, 5 kb, 6 kb, 7 kb, 9 kb, 10 kb, 20 kb, 30 kb, 40 kb, 50 kb, 60 kb, 70 kb, 80 kb, 90 kb, 100 kb, 500 kb, or 1 Mb. Having a size cutoff may result in a higher subread depth, either of which may increase the accuracy of the methylation detection. In some embodiments, the method may include fractionating the DNA molecules for certain sizes prior to sequencing the DNA molecules.

The plurality of sample nucleic acid molecules may align to a plurality of genomic regions. For each genomic region of the plurality of genomic regions, a number of sample nucleic acid molecules may be aligned to the genomic region. The number of sample nucleic acid molecules may be greater than a cutoff number. The cutoff number may be a subread depth cutoff. The subread depth cutoff number may be 1x, 10x, 30x, 40x, 50x, 60x, 70x, 80x, 900x, 100x, 200x, 300x, 400x, 500x, 600x, 700x, or 800x, or within any range between and including any of these numbers. The subread depth cutoff number may be determined to improve or to optimize accuracy. The subread depth cutoff number may be related to the number of the plurality of genomic regions. For example, a higher subread depth cutoff number, a lower number of the plurality of genomic regions.

The methylation may be determined to be present at one or more nucleotides. A classification of a disorder may be determined using the presence of the methylation at one or more nucleotides. The classification of the disorder may include using the number of methylations. The number of methylations may be compared to a threshold. The comparison may be used to determine whether a site or region is hypermethylated or hypomethylated. Alternatively or additionally, the classification may include the location of the one or more methylations. The location of the one or more methylations may be determined by aligning sequence reads of a nucleic acid molecule to a reference genome. The disorder may be determined if certain locations known to be correlated with the disorder are shown to have the methylation. For example, a pattern of methylated sites may be compared to a reference pattern for a disorder, and the determination of the disorder may be based on the comparison. A match with the reference pattern or a substantial match (e.g., 80%, 90%, or 95% or more) with the reference pattern may indicate the disorder or a high likelihood of the disorder. The disorder may be cancer or any disorder (e.g., pregnancy-associated disorder, autoimmune disease) described herein.

A statistically significant number of nucleic acid molecules can be analyzed so as to provide an accurate determination for a disorder, tissue origin, or clinically-relevant DNA fraction. In some embodiments, at least 1,000 nucleic acid molecules are analyzed. In other embodiments, at least 10,000 or 50,000 or 100,000 or 500,000 or 1,000,000 or 5,000,000 nucleic acid molecules, or more, can be analyzed. As a further example, at least 10,000 or 50,000 or 100,000 or 500,000 or 1,000,000 or 5,000,000 sequence reads can be generated.

The method may include determining that the classification of the disorder is that the subject has the disorder. The classification may include a level of the disorder, using the number of methylations and/or the sites of the methylations.

A clinically-relevant DNA fraction, a fetal methylation profile, a maternal methylation profile, a presence of an imprinting gene region, a tissue of origin (e.g., from a sample containing a mixture of different cell types), or a location of a CTCF binding site may be determined using the presence of the methylation at one or more nucleotides. Whether a site or region is hypermethylated or hypomethylated may indicate the origin of a fragment. For example, certain genomic loci are known to be hypermethylated in cell-free fetal DNA compared to cell-free maternal DNA. Clinically-relevant DNA fraction includes, but is not limited to, fetal DNA fraction, tumor DNA fraction (e.g., from a sample containing a mixture of tumor cells and non-tumor cells), and transplant DNA fraction (e.g., from a sample containing a mixture of donor cells and recipient cells).

The method may further include treating the disorder. Treatment can be provided according to a determined level of the disorder, the identified methylations, and/or the tissue of origin (e.g., of tumor cells isolated from the circulation of a cancer patient). For example, an identified methylation can be targeted with a particular drug or chemotherapy. The tissue of origin can be used to guide a surgery or any other form of treatment. And, the level of disorder can be used to determine how aggressive to be with any type of treatment.

Embodiments may include treating the disorder in the patient after determining the level of the disorder in the patient. Treatment may include any suitable therapy, drug, chemotherapy, radiation, or surgery, including any treatment described in a reference mentioned herein. Information on treatments in the references are incorporated herein by reference.

Process 2100 may include additional implementations, such as any single implementation or any combination of implementations described below and/or in connection with one or more other processes described elsewhere herein.

Although FIG. 21 shows example blocks of process 2100, in some implementations, process 2100 may include additional blocks, fewer blocks, different blocks, or differently arranged blocks than those depicted in FIG. 21 Additionally, or alternatively, two or more of the blocks of process 2100 may be performed in parallel.

### II. Use of Kinetic Signals from Adaptor Sequences

Sequence data generated from PacBio SMRT-seq is typically trimmed to remove the ligated adaptor sequences before downstream analysis. Measurement windows described herein may have a minimum size and may use a number of nucleotides upstream and downstream of a target nucleotide for analysis. Target nucleotide sites (e.g., CpG sites) that are at the end or close to an end of a DNA molecule may not have sufficient upstream or downstream nucleotides to construct such a measurement window of kinetic signal data for methylation analysis. Hence, the regions that contain CpG sites near ends of DNA fragments typically would be classified as no call regions. On average, 20.5% of cell-free DNA molecules contained at least one CpG falling within the no call regions (i.e., within 10 nt of an end). The use of measurement windows may therefore result in data loss or reduce the informativeness of methylation analysis.

Embodiments described herein may use the kinetic signals (e.g., IPDs and PWs) derived from the trimmed adaptor sequences to construct a complete measurement window for a target nucleotide (e.g., CpG site) close to the ends of a fragment. Hence, those CpG sites close to the ends, which are otherwise not analyzable, may be analyzed.

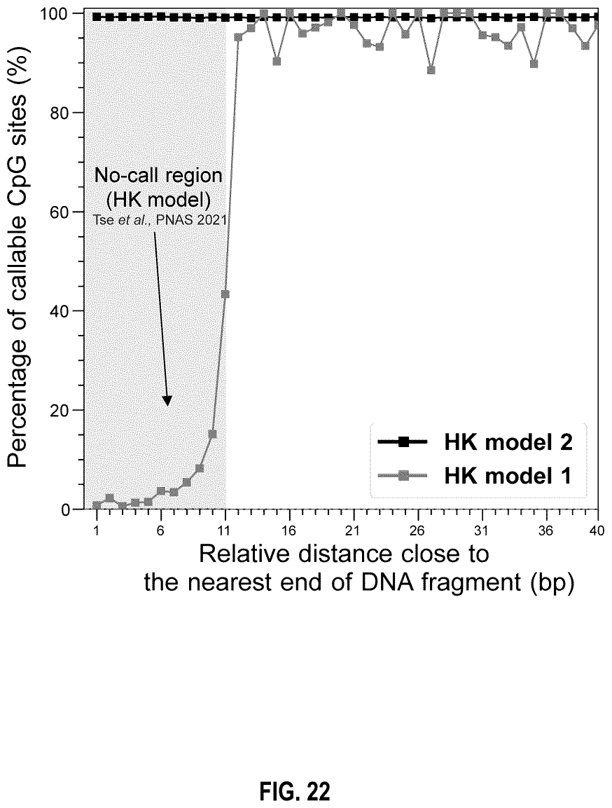

FIG. 22 shows a graph of the callable CpG sites versus distance to the nearest end. The x-axis is the relative distance to the nearest end of the DNA fragment in base pairs. The y-axis is the percentage of callable CpG sites. FIG. 22 shows the rapid reduction in the percentage of callable CpG sites close to fragment ends within a nucleotide distance of 11 nt using HK model 1. The gray rectangle indicates the no-call region of HK model 1. To overcome the issue, HK model 2 made use of kinetic signals retrieved from sequencing adaptors to facilitate the methylation analysis of CpGs proximal to the fragment ends. The percentage of callable CpG sites in HK model 2 bounced back to nearly 100%.

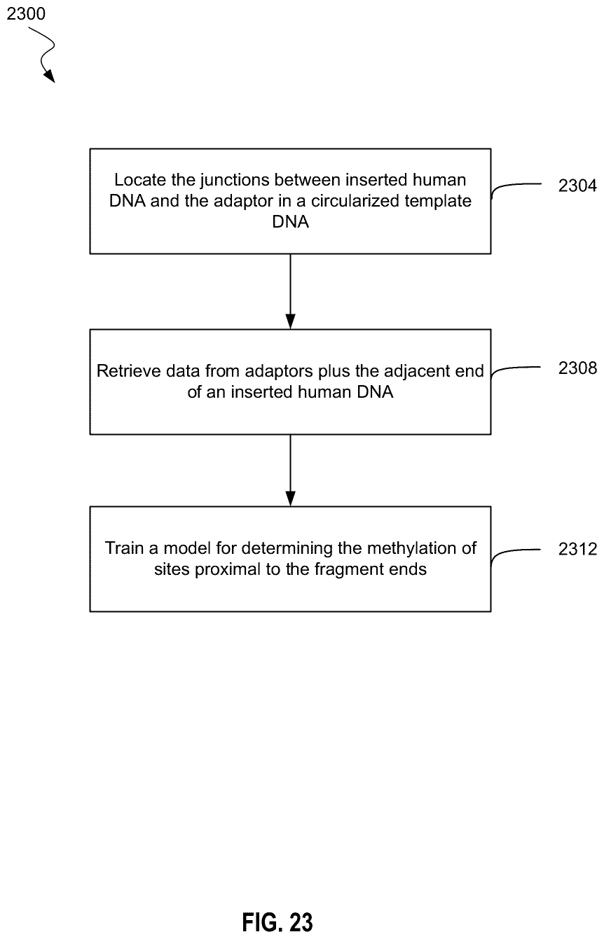

FIG. 23 shows a workflow 2300 for using adaptor sequences to analyze methylation of a site.

At block 2304, the junctions between inserted human DNA and the adaptor in a circularized template DNA are located. Known adaptor sequences may be identified using pairwise alignment.

At block 2308, data from the adaptors and the adjacent end of the inserted human DNA are retrieved. The data may include kinetic signals (e.g., IPD, PW) and the identities of the nucleotides. These kinetic signals may be any kinetic features described herein, including sections I.A.1 and II.A.

At block 2312, a model is trained for determining the methylation of sites near the fragment ends. Training the model may include a training data set with target nucleotides near (e.g., 10 nt) or at the end. The training data set may also include target nucleotides away from the end (e.g., farther than 10 nt from the closest end).

The trained model may then be used to analyze methylation of sites near fragment ends. In some embodiments, the trained model may be dedicated to nucleotides near or at the end of a nucleic acid molecule. The trained model may be used only after comparing the position of the target nucleotide to a threshold (e.g., 10 nt from an end) and if the position is under the threshold, then the trained model would be used.

FIG. 24 is a graph of the performance of the EMA model for determining methylation status of CpG sites within 10 nt of the 5′ end of the DNA fragment. The x-axis shows specificity. The y-axis shows sensitivity. The model using data from adaptor sequences achieves an AUC of 0.97 for distinguishing the methylation from unmethylation for those CpG sites within a distance of 10 nt relative to the 5′ end.

### A. Example Method of Model Training

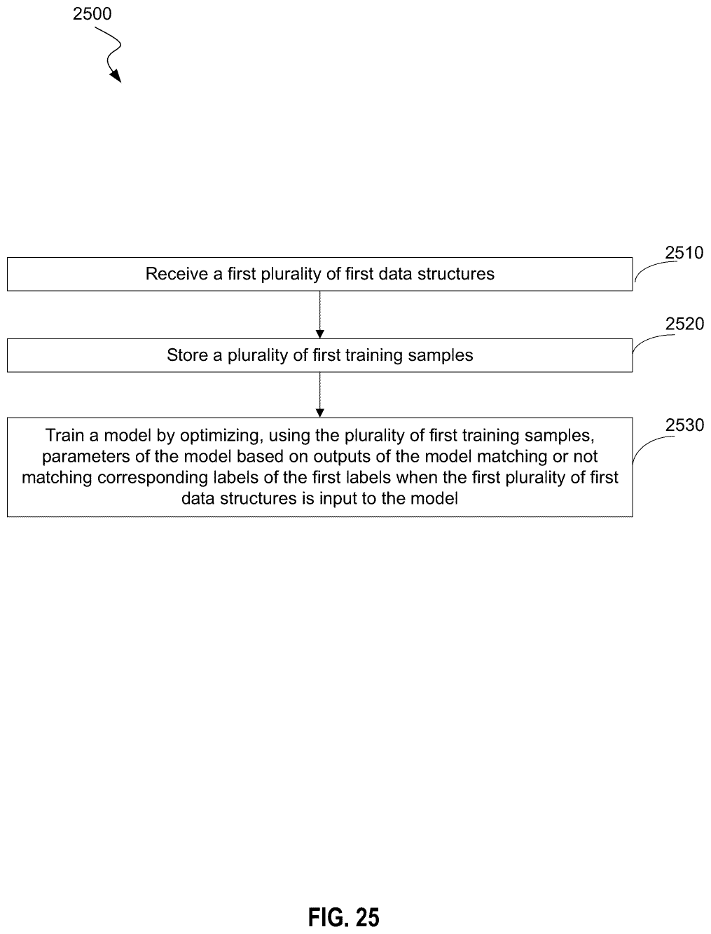

FIG. 25 is a flowchart of an example process 2500 for detecting a methylation of a nucleotide in a nucleic acid molecule. Process 2500 may be used for training a model using nucleic acid molecules with an adaptor. In some implementations, one or more process blocks of FIG. 25 may be performed by system 2700 or any system described herein. The methylation may be any methylation described herein, including 5 mC (5-methylcytosine) or 6 mA (N6-methyladenine).

At block 2510, a first plurality of first data structures is received. Each first data structure of the first plurality of first data structures may correspond to a respective window of nucleotides sequenced in a respective nucleic acid molecule of a plurality of first nucleic acid molecules. Each of the first nucleic acid molecules may be sequenced by measuring pulses in a signal corresponding to the nucleotides. Each first nucleic acid molecule may include a training sample nucleic acid molecule and a first adaptor having a known sequence. In some embodiments, the known sequence may be the identity of the single nucleotide ligated to the end of the first nucleic acid molecule. The methylation may have a known first state in a nucleotide at a target position in a portion of each window of each first nucleic acid molecule corresponding to the training sample nucleic acid molecule. Each first data structure may include values for one or more signal properties. The plurality of known sequences corresponding to the first adaptors of the plurality of first nucleic acid molecules may be the same or different.

The signal may be an optical signal or an electrical signal. The optical signal may be from single molecule, real-time sequencing. The electrical signal may be from nanopore sequencing. The signal may be any signal described herein, including with process 2000.

The one or more signal properties may include the sequence context. The one or more signal properties may include an identity of the nucleotide for each nucleotide within each window. The one or more signal properties may also include, for each nucleotide within each window, a position of the nucleotide within the sample nucleic acid molecule, a width of a pulse corresponding to the nucleotide, and/or an interpulse duration representing a time between the pulse corresponding to the nucleotide and a pulse corresponding to a neighboring nucleotide. The signal properties may be the signal properties described with process 2000 or any signal properties described herein.

A subset of the windows may include at least 1, 2, 3, 4, 5, 6, 7, 8 or more nucleotides in the adaptor.

The nucleotides within each window may be determined using a circular consensus sequence and without alignment of the sequenced nucleotides to a reference genome.

Block 2510 may be performed similar to block 2010.

At block 2520, a plurality of first training samples is stored. Each first training sample may include one of the first plurality of first data structures and a first label indicating the first state of the nucleotide at the target position. Block 2520 may be performed similar to block 2020.

At block 2530, a model is trained by optimizing, using the plurality of first training samples, parameters of the model based on outputs of the model matching or not matching corresponding labels of the first labels when the first plurality of first data structures is input to the model. An output of the model may specify whether the nucleotide at the target position in the respective window has the methylation.

In some embodiments, the training sample nucleic acid molecule may have two adaptors. The known sequence is a first known sequence. Each first nucleic acid molecule may include the first adaptor at a first end. Each first nucleic acid molecule may include a second adaptor at a second end. The second adaptor may have a second known sequence. Each training sample nucleic acid molecule may have the first adaptor at one end and the second adaptor at the other end. A subset of the windows may include at least one nucleotide in the second adaptor.

In some embodiments, the training sample nucleic acid molecules may be limited to nucleotides having a target position within some distance from the closest end of the first nucleic acid molecule. For example, the target position may be within 1, 2, 3, 4, 5, 6, 7, 8, 9, 10, 10 to 15, or 15 to 20 nucleotides from an end. In some embodiments, the training sample nucleic acid molecule may include nucleotides that are not restricted to certain positions (e.g., molecules may have a target position greater than 10 nt from an end).

The model may include a convolutional neural network (CNN). The CNN may include a set of convolutional filters configured to filter the first plurality of data structures and optionally the second plurality of data structures. The filter may be any filter described herein. The number of filters for each layer may be from 10 to 20, 20 to 30, 30 to 40, 40 to 50, 50 to 60, 60 to 70, 70 to 80, 80 to 90, 90 to 100, 100 to 150, 150 to 200, or more. The kernel size for the filters can be 2, 3, 4, 5, 6, 7, 8, 9, 10, 11, 12, 13, 14, 15, from 15 to 20, from 20 to 30, from 30 to 40, or more. The CNN may include an input layer configured to receive the filtered first plurality of data structures and optionally the filtered second plurality of data structures. The CNN may also include a plurality of hidden layers including a plurality of nodes. The first layer of the plurality of hidden layers coupled to the input layer. The CNN may further include an output layer coupled to a last layer of the plurality of hidden layers and configured to output an output data structure. The output data structure may include the properties.

In some embodiments, the model may include a recurrent neural network (RNN). The RNN may be in place of the CNN. The model may include a supervised learning model. Supervised learning models may include different approaches and algorithms including analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, Nearest Neighbor Algorithm, probably approximately correct learning (PAC) learning, ripple down rules, a knowledge acquisition methodology, symbolic machine learning algorithms, subsymbolic machine learning algorithms, support vector machines, Minimum Complexity Machines (MCM), random forests, ensembles of classifiers, ordinal classification, data pre-processing, handling imbalanced datasets, statistical relational learning, or Proaftn, a multicriteria classification algorithm The model may linear regression, logistic regression, deep recurrent neural network (e.g., long short term memory, LSTM), Bayes classifier, hidden Markov model (HMM), linear discriminant analysis (LDA), k-means clustering, density-based spatial clustering of applications with noise (DBSCAN), random forest algorithm, support vector machine (SVM), or any model described herein.

As part of training a machine learning model, the parameters of the machine learning model (such as weights, thresholds, e.g., as may be used for activation functions in neural networks, etc.) can be optimized based on the training samples (training set) to provide an optimized accuracy in classifying the methylation of the nucleotide at the target position. Various form of optimization may be performed, e.g., backpropagation, empirical risk minimization, and structural risk minimization. A validation set of samples (data structure and label) can be used to validate the accuracy of the model. Cross-validation may be performed using various portions of the training set for training and validation. The model can comprise a plurality of submodels, thereby providing an ensemble model. The submodels may be weaker models that once combined provide a more accurate final model.

In some embodiments, training may include one or more transformer layers and may be performed similar to blocks 2030-2080.

Process 2500 may include additional implementations, such as any single implementation or any combination of implementations described herein and/or in connection with one or more other processes described elsewhere herein.

Although FIG. 25 shows example blocks of process 2500, in some implementations, process 2500 may include additional blocks, fewer blocks, different blocks, or differently arranged blocks than those depicted in FIG. 25. Additionally, or alternatively, two or more of the blocks of process 2500 may be performed in parallel.

### B. Example Method of Detecting Methylations

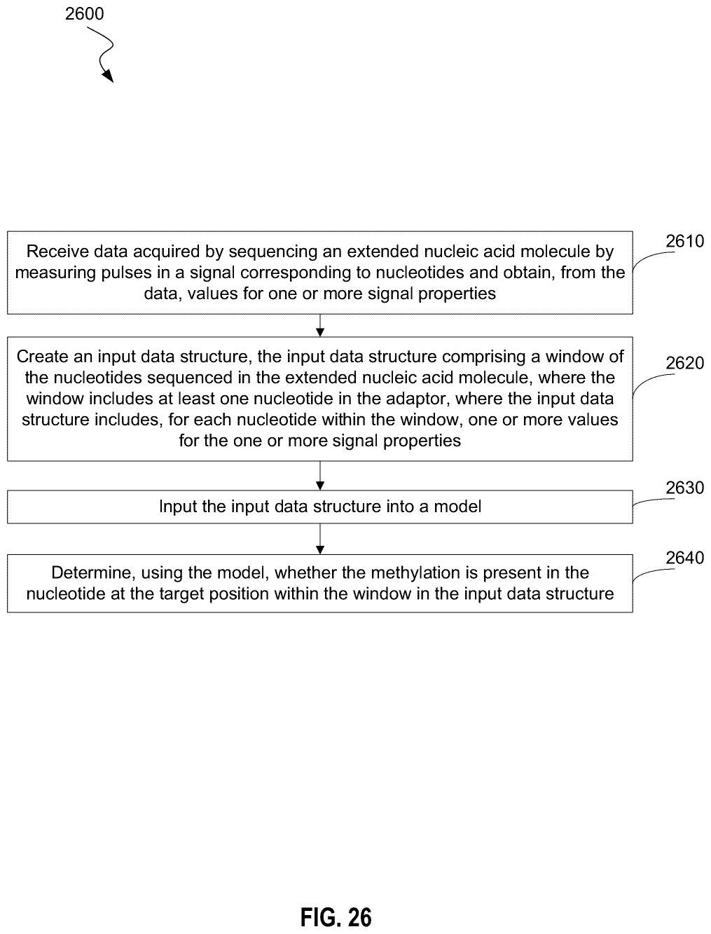

FIG. 26 is a flowchart of an example process 2600 for detecting a methylation of a nucleotide in a nucleic acid molecule. In some implementations, one or more process blocks of FIG. 26 may be performed by system 2700 or any system described herein. The methylation may be any methylation described herein, including 5 mC (5-methylcytosine) or 6 mA (N6-methyladenine).

At block 2610, data acquired by sequencing an extended nucleic acid molecule by measuring pulses in a signal corresponding to nucleotides is received. The signal may be an optical signal or an electrical signal. The extended nucleic acid molecule may include a sample nucleic acid molecule and an adaptor. The adaptor may have a known sequence. Values may be obtained from the data for one or more signal properties. The signal properties may be any signal properties described herein, including the signal properties described with process 2000 or block 2510. Block 2610 may be performed similar to block 2110.

In some embodiments, process 2600 may include ligating an adaptor onto the sample nucleic acid molecule. In embodiments, the extended nucleic acid molecule may be sequenced with nanopore sequencing. In other embodiments, the extended nucleic acid molecule may be sequenced with single molecule real-time sequencing.

At block 2620, an input data structure is created. The input data structure may include a window of the nucleotides sequenced in the extended nucleic acid molecule. The window may include at least one nucleotide in the adaptor. The window may include at least at least 1, 2, 3, 4, 5, 6, 7, 8 or more nucleotides in the adaptor. The input data structure may include, for each nucleotide within the window, one or more values for the one or more signal properties.

The nucleotides within the window may be determined using a circular consensus sequence and without alignment of the sequenced nucleotides to a reference genome. The window of the input data structure may have similar properties as the window of each first data structure in process 2000.

At block 2630, the input data structure is inputted into a model. The model may be trained by process 2500 or any method described herein. The known sequence may be the same or different from the sequences of adaptors in the training set. The model may include the framework described with FIG. 1 in section I.A.

In some embodiments, the position of the target nucleotide may be determined, and the distance from the position to the closest end may be calculated. The distance may be compared to a threshold. If the distance is less than a certain threshold (e.g., 1, 2, 3, 4, 5, 6, 7, 8, 9, 10, 10 to 15, or 15 to 20 nucleotides), then the input data structure is inputted into the model. If the distance is greater than a certain threshold, then the input data structure may be inputted into a second model, which is not trained with measurement windows including nucleotides with adaptors.

At block 2640, whether the methylation is present in the nucleotide at the target position within the window in the input data structure is determined using the model. Block 2640 may be performed similar to block 2140.

In some embodiments, process 2600 may further include determining whether the methylation is more likely a first type or a second type. For example, the first type may be one of 5 mC, 5 hmC, 6 mA, or any methylation described herein. The second type may be different from the first type. Process 2600 may determine not just that a methylation is present but the type of methylation present (e.g., as described with FIGS. 8A, 8B, and 8C).

The input data structure may be one input data structure of a plurality of input data structures as described with process 2100. The methylation determinations may be used as described with process 2100.

In some embodiments, the extended nucleic acid molecule may include two adaptors. The adaptor is a first adaptor. The known sequence may be a first known sequence. The extended nucleic acid molecule may include a first adaptor at a first end. The extended nucleic acid molecule may include a second adaptor at a second end. The second adaptor may have a second known sequence. The window may include at least one nucleotide in the second adaptor.

Process 2600 may include additional implementations, such as any single implementation or any combination of implementations described herein and/or in connection with one or more other processes described elsewhere herein.

Although FIG. 26 shows example blocks of process 2600, in some implementations, process 2600 may include additional blocks, fewer blocks, different blocks, or differently arranged blocks than those depicted in FIG. 26. Additionally, or alternatively, two or more of the blocks of process 2600 may be performed in parallel.

### III. EXAMPLE SYSTEMS

FIG. 27 illustrates a measurement system 2700 according to an embodiment of the present disclosure. The system as shown includes a sample 2705, such as cell-free nucleic acid molecules (e.g., DNA and/or RNA) within an assay device 2710, where an assay 2708 can be performed on sample 2705. For example, sample 2705 can be contacted with reagents of assay 2708 to provide a signal (e.g., an intensity signal) of a physical characteristic 2715 (e.g., sequence information of a cell-free nucleic acid molecule). An example of an assay device can be a flow cell that includes probes and/or primers of an assay or a tube through which a droplet moves (with the droplet including the assay). Another example of an assay device is a sequencing device. Physical characteristic 2715 (e.g., a fluorescence intensity, a voltage, or a current), from the sample is detected by detector 2720. Detector 2720 can take a measurement at intervals (e.g., periodic intervals) to obtain data points that make up a data signal. In one embodiment, an analog-to-digital converter converts an analog signal from the detector into digital form at a plurality of times.

Assay device 2710 and detector 2720 can form an assay system, e.g., a sequencing system that performs sequencing according to embodiments described herein. A data signal 2725 is sent from detector 2720 to logic system 2730. As an example, data signal 2725 can be used to determine sequences and/or locations in a reference genome of nucleic acid molecules (e.g., DNA and/or RNA). Data signal 2725 can include various measurements made at a same time, e.g., different colors of fluorescent dyes or different electrical signals for different molecule of sample 2705, and thus data signal 2725 can correspond to multiple signals. Data signal 2725 may be stored in a local memory 2735, an external memory 2740, or a storage device 2745. The assay system can be comprised of multiple assay devices and detectors.

Logic system 2730 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 2730 and the other components may be part of a stand-alone or network connected computer system, or they may be directly attached to or incorporated in a device (e.g., a sequencing device) that includes detector 2720 and/or assay device 2710. Logic system 2730 may also include software that executes in a processor 2750. Logic system 2730 may include a computer readable medium storing instructions for controlling measurement system 2700 to perform any of the methods described herein. For example, logic system 2730 can provide commands to a system that includes assay device 2710 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay.

Measurement system 2700 may also include a treatment device 2760, which can provide a treatment to the subject. Treatment device 2760 can determine a treatment and/or be used to perform a treatment. Examples of such treatment can include surgery, radiation therapy, chemotherapy, immunotherapy, targeted therapy, hormone therapy, and stem cell transplant. Logic system 2730 may be connected to treatment device 2760, e.g., to provide results of a method described herein. The treatment device may receive inputs from other devices, such as an imaging device and user inputs (e.g., to control the treatment, such as controls over a robotic system).

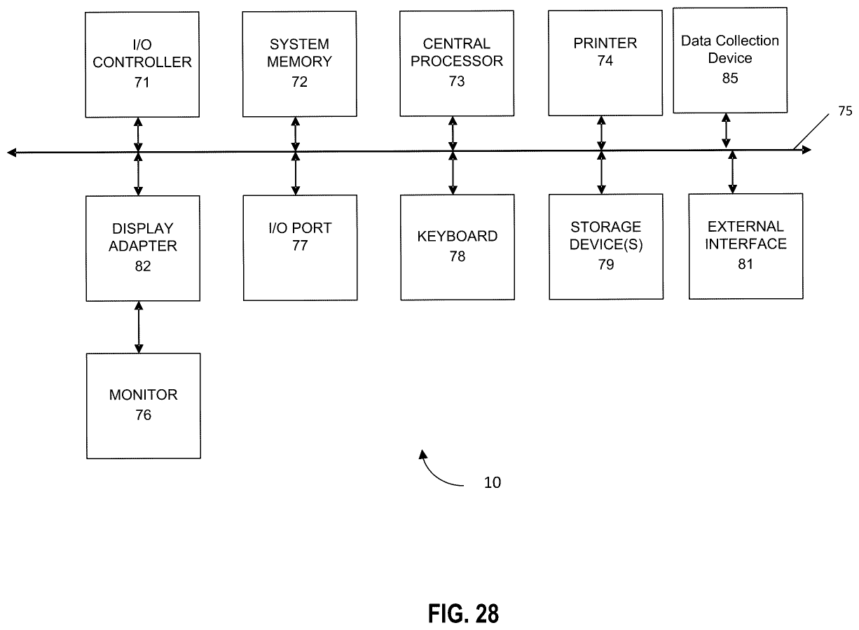

Any of the computer systems mentioned herein may utilize any suitable number of subsystems. Examples of such subsystems are shown in FIG. 28 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 28 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, Lightning). For example, I/O port 77 or external interface 81 (e.g. Ethernet, Wi-Fi, etc.) can be used to connect computer system 1500 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g., an application specific integrated circuit or field programmable gate array) and/or using computer software with a generally programmable processor in a modular or integrated manner. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive, or an optical medium such as a compact disk (CD) or DVD (digital versatile disk) or Blu-ray disk, flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order that is logically possible. Additionally, portions of these steps may be used with portions of other steps from other methods. Also, all or portions of a step may be optional. Additionally, any of the steps of any of the methods can be performed with modules, units, circuits, or other means of a system for performing these steps.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The above description of example embodiments of the present disclosure has been presented for the purposes of illustration and description and are set forth so as to provide those of ordinary skill in the art with a complete disclosure and description of how to make and use embodiments of the present disclosure. It is not intended to be exhaustive or to limit the disclosure to the precise form described nor are they intended to represent that the experiments are all or the only experiments performed. Although the disclosure has been described in some detail by way of illustration and example for purposes of clarity of understanding, it is readily apparent to those of ordinary skill in the art in light of the teachings of this disclosure that certain changes and methylations may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the disclosure being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within embodiments of the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

All patents, patent applications, publications, and descriptions mentioned herein are hereby incorporated by reference in their entirety for all purposes as if each individual publication or patent were specifically and individually indicated to be incorporated by reference and are incorporated herein by reference to disclose and describe the methods and/or materials in connection with which the publications are cited. None is admitted to be prior art.

### IV. REFERENCES

1. Tse O Y O, et al. Genome-wide detection of cytosine methylation by single molecule real-time sequencing. Proc Natl Acad Sci USA 118, (2021).
2. Yu S C Y, et al. Single-molecule sequencing reveals a large population of long cell-free DNA molecules in maternal plasma. Proc Natl Acad Sci USA 118, (2021).
3. Choy L Y L, et al. Single-Molecule Sequencing Enables Long Cell-Free DNA Detection and Direct Methylation Analysis for Cancer Patients. Clin Chem 68, 1151-1163 (2022).
4. Yu S C Y, et al. Comparison of Single Molecule, Real-Time Sequencing and Nanopore Sequencing for Analysis of the Size, End-Motif, and Tissue-of-Origin of Long Cell-Free DNA in Plasma. Clin Chem 69, 168-179 (2023).
5. Lau B T, et al. Single-molecule methylation profiles of cell-free DNA in cancer with nanopore sequencing. Genome Med 15, 33 (2023).
6. Pastor W A, et al. Genome-wide mapping of 5-hydroxymethylcytosine in embryonic stem cells. Nature 473, 394-397 (2011).
7. Wen L, et al. Whole-genome analysis of 5-hydroxymethylcytosine and 5-methylcytosine at base resolution in the human brain. Genome Biol 15, R49 (2014).
8. Cui X L, et al. A human tissue map of 5-hydroxymethylcytosines exhibits tissue specificity through gene and enhancer modulation. Nat Commun 11, 6161 (2020).
9. Li W, et al. 5-Hydroxymethylcytosine signatures in circulating cell-free DNA as diagnostic biomarkers for human cancers. Cell Res 27, 1243-1257 (2017).
10. Song C X, et al. 5-Hydroxymethylcytosine signatures in cell-free DNA provide information about tumor types and stages. Cell Res 27, 1231-1242 (2017).
11. Luo G Z, Blanco M A, Greer E L, He C, Shi Y. DNA N(6)-methyladenine: a new epigenetic mark in eukaryotes? Nat Rev Mol Cell Biol 16, 705-710 (2015).
12. Stergachis A B, Debo B M, Haugen E, Churchman L S, Stamatoyannopoulos JA. Single-molecule regulatory architectures captured by chromatin fiber sequencing. Science 368, 1449-1454 (2020).
13. Xu C, Corces V G. Nascent DNA methylome mapping reveals inheritance of hemimethylation at CTCF/cohesin sites. Science 359, 1166-1170 (2018).
14. Tamanaha E, Guan S, Marks K, Saleh L. Distributive Processing by the Iron(II)/alpha-Ketoglutarate-Dependent Catalytic Domains of the TET Enzymes Is Consistent with Epigenetic Roles for Oxidized 5-Methylcytosine Bases. J Am Chem Soc 138, 9345-9348 (2016).
15. Beaulaurier J, Schadt E E, Fang G. Deciphering bacterial epigenomes using modern sequencing technologies. Nat Rev Genet 20, 157-172 (2019).
16. Kong Y, et al. Critical assessment of DNA adenine methylation in eukaryotes using quantitative deconvolution. Science 375, 515-522 (2022).
17. McIntyre A B R, et al. Single-molecule sequencing detection of N6-methyladenine in microbial reference materials. Nat Commun 10, 579 (2019).
18. Yang S, Wang Y, Chen Y, Dai Q. MASQC: Next Generation Sequencing Assists Third Generation Sequencing for Quality Control in N6-Methyladenine DNA Identification. Front Genet 11, 269 (2020).
19. Jiang P, et al. Detection and characterization of jagged ends of double-stranded DNA in plasma. Genome Res 30, 1144-1153 (2020).
20. Vaswani A, et al. Attention is all you need. Advances in neural information processing systems 30, (2017).
21. Ito S, et al. Tet proteins can convert 5-methylcytosine to 5-formylcytosine and 5-carboxylcytosine. Science 333, 1300-1303 (2011).

1. A method for detecting a methylation of a nucleotide in a nucleic acid molecule, the method comprising:
receiving data acquired by sequencing a sample nucleic acid molecule by measuring pulses in a signal corresponding to nucleotides of the sample nucleic acid molecule and obtaining, from the data, values for one or more signal properties;
creating an input data structure, the input data structure comprising a window around a target position of the nucleotides sequenced in the sample nucleic acid molecule, wherein the input data structure includes, for each nucleotide within the window, one or more values for the one or more signal properties;
inputting the input data structure into a model, wherein the model is a machine learning model, the model trained by:
receiving a first plurality of first data structures, each first data structure of the first plurality of first data structures corresponding to a respective window around a respective target position of nucleotides sequenced in a respective nucleic acid molecule of a plurality of first nucleic acid molecules, wherein each of the first nucleic acid molecules is sequenced by measuring pulses in the signal corresponding to the nucleotides, wherein the methylation has a known first state in a nucleotide at the respective target position in each window of each first nucleic acid molecule, each first data structure comprising values for the same properties as the input data structure,
storing a plurality of first training samples, each including one of the first plurality of first data structures and a first label indicating the first state of the nucleotide at the respective target position,
filtering the first plurality of first data structures through one or more convolutional layers to obtain a plurality of convolutional matrices,
applying a transformer layer to the plurality of convolutional matrices to obtain transformer matrices, wherein applying the transformer layer to a convolutional matrix includes generating a plurality of attention scores that quantify a relevance among positions of the convolutional matrix,
generating methylation probabilities at respective target positions of the first plurality of first data structures using the transformer matrices,
determining outputs using the methylation probabilities, and
optimizing, using the plurality of first training samples, parameters of the model based on the outputs of the model matching or not matching corresponding labels of the first labels when the first plurality of first data structures is input to the model, wherein an output of the model specifies whether the nucleotide at the respective target position in the respective window has the methylation, wherein the parameters of the model include the plurality of attention scores,
determining, using the model, whether the methylation is present in the nucleotide at the target position within the window in the input data structure.

2. The method of claim 1, wherein the signal is an optical signal or an electrical signal.

3. The method of claim 1, wherein the one or more signal properties include:
for each nucleotide within the window:
an identity of the nucleotide.

4. The method of claim 3, wherein the one or more signal properties further include:
for each nucleotide within the window:
a position of the nucleotide within the sample nucleic acid molecule,
a width of a pulse corresponding to the nucleotide, or
an interpulse duration representing a time between the pulse corresponding to the nucleotide and a pulse corresponding to a neighboring nucleotide.

5. The method of claim 1, wherein generating the plurality of attention scores comprises using a plurality of multiple-head self-attentions.

6. The method of claim 1, wherein generating the methylation probabilities comprises applying one or more neural network layers to the transformer matrices.

7. The method of claim 6, wherein applying the one or more neural network layers comprises performing multiplication by weights or additions by biases.

8. The method of claim 1, wherein the respective convolutional results have lower dimensionality than the respective first data structure.

9. The method of claim 1, wherein the methylation is 5 mC (5-methylcytosine).

10. The method of claim 1, wherein the methylation is 6 mA (N6-methyladenine).

11. The method of claim 1, wherein the window comprises 13 consecutive nucleotides.

12. The method of claim 1, wherein the window of the input data structure has a different number of consecutive nucleotides upstream of the nucleotide at the target position than the number of consecutive nucleotides downstream of the nucleotide at the target position.

13. The method of claim 1, wherein the window of the input data structure comprises 21 consecutive nucleotides upstream of the nucleotide at the target position and 21 consecutive nucleotides downstream of the nucleotide at the target position.

14. The method of claim 1, wherein:
the data is acquired by sequencing an extended nucleic acid molecule,
the extended nucleic acid molecule comprises the sample nucleic acid molecule and an adaptor,
the adaptor has a known sequence, and
the window of nucleotides includes at least one nucleotide in the adaptor.

15. The method of claim 1, wherein determining whether the methylation is present comprises:
determining the methylation is present; and
determining the methylation is a first type from among a plurality of types.

16. The method of claim 15, wherein each type of the plurality of types is selected from the group consisting of 5 mC, 5 hmC, and 6 mA.

17. The method of claim 1, wherein the sample nucleic acid molecule is single-stranded.

18. The method of claim 17, wherein the plurality of first nucleic acid molecules comprises single-stranded nucleic acid molecules.

19. A method for detecting a methylation of a nucleotide in a nucleic acid molecule, the method comprising:
receiving a first plurality of first data structures, each first data structure of the first plurality of first data structures corresponding to a respective window of positions around a respective target position of nucleotides sequenced in a respective nucleic acid molecule of a plurality of first nucleic acid molecules, wherein each of the first nucleic acid molecules is sequenced by measuring pulses in a signal corresponding to the nucleotides, wherein the methylation has a known first state in a nucleotide at the respective target position in each window of each first nucleic acid molecule, each first data structure comprising values for one or more signal properties at positions within the respective window;
storing a plurality of first training samples, each first training sample including one of the first plurality of first data structures and a first label indicating the first state of the nucleotide at the respective target position; and
training a model by:
filtering the first plurality of first data structures through one or more convolutional layers to obtain a plurality of convolutional matrices,
applying a transformer layer to the plurality of convolutional matrices to obtain transformer matrices, wherein applying the transformer layer to a convolutional matrix includes generating a plurality of attention scores that quantify a relevance among positions of the convolutional matrices,
generating methylation probabilities at respective target positions of the first plurality of first data structures using the transformer matrices,
determining outputs using the methylation probabilities, and
optimizing, using the plurality of first training samples, parameters of the model based on the outputs of the model matching or not matching corresponding labels of the first labels when the first plurality of first data structures is input to the model, wherein an output of the model specifies whether the nucleotide at the respective target position in the respective window has the methylation, wherein the parameters of the model include the plurality of attention scores.

20-52. (canceled)

53. A method for detecting a methylation of a nucleotide in a nucleic acid molecule, the method comprising:
receiving a first plurality of first data structures, each first data structure of the first plurality of first data structures corresponding to a respective window around a respective target position of nucleotides sequenced in a respective nucleic acid molecule of a plurality of first nucleic acid molecules, wherein each of the first nucleic acid molecules is sequenced by measuring pulses in a signal corresponding to the nucleotides, wherein each first nucleic acid molecule comprises a training sample nucleic acid molecule and a first adaptor having a known sequence, wherein the methylation has a known first state in a nucleotide at the respective target position in a portion of each window of each first nucleic acid molecule corresponding to the training sample nucleic acid molecule, each first data structure comprising values for one or more signal properties;
storing a plurality of first training samples, each including one of the first plurality of first data structures and a first label indicating the first state of the nucleotide at the respective target position, and
training a model by optimizing, using the plurality of first training samples, parameters of the model based on outputs of the model matching or not matching corresponding labels of the first labels when the first plurality of first data structures is input to the model, wherein an output of the model specifies whether the nucleotide at the respective target position in the respective window has the methylation.

54-85. (canceled)
